(19) United States
(12) Patent Application Publication
(10) Pub. No.: US 2010/0152811 A1
(43) Pub. Date: Jun. 17, 2010

(54) NERVE REGENERATION SYSTEM AND LEAD DEVICES ASSOCIATED THEREWITH

(76) Inventor: Christopher J. Flaherty, Topsfield, MA (US)

Correspondence Address: PANDISCIO & PANDISCIO, P.C., 470 TOTTEN POND ROAD, WALTHAM, MA, 02451-1914, US

(21) Appl. No.: 12/305,225

(22) PCT Filed: Jun. 29, 2007
(86) PCT No.: PCT/US07/72496

§ 371 Date: Nov. 30, 2009

Related U.S. Application Data
(60) Provisional application No. 60/817,342, filed on Jun. 30, 2006.

(51) Int. Cl. A61N 1/05

(52) U.S. Cl. 607/50

(57) ABSTRACT

Various systems and methods for promoting nerve regeneration are disclosed. The system may include an elongated lead configured to be implanted within a patient's body. The system may also include a plurality of electrodes disposed along the elongated lead and configured to deliver electric stimulation to an area of a patient's body. The plurality of electrodes may comprise at least one transmitting electrode in communication with the controller, wherein the at least one transmitting electrode is configured to transmit an electric signal to one or more other electrodes. The controller may be configured to control operation of the at least one transmitting electrode.

This application claims the benefit of priority under 35 U.S.C. §119(e) of U.S. Provisional Application No. 60/817,342, filed Jun. 30, 2006, which is incorporated by reference herein in its entirety.

### FIELD OF THE INVENTION

The present disclosure relates generally to systems and methods for causing nerve cells to regenerate and, more particularly, to systems and methods for promoting nerve regeneration in the central and peripheral nervous systems of mammals.

### DESCRIPTION OF RELATED ART

The central nervous system, including the brain, is the primary control system of a body, communicating with one or more parts of the body via a complicated system of interconnected nerves. Nerves are cable-like bundles of axons that carry electrical signals and impulses between one or more neurons and the central nervous system. Thus, nerves play a critical role in communicating sensory and stimulatory signals between various parts of the body (e.g., muscles, organs, glands, etc.) and the central nervous system.

Nerves may be damaged or severed either through trauma or disease. Damaged or severed nerves may inhibit the central nervous system's ability to receive sensory and stimulatory data from individual neurons, potentially limiting the nervous system's control over the body. For example, severe nerve damage may lead to paralysis, such as paraplegia or quadriplegia.

In the case of the peripheral nervous system (i.e., the portion of the nervous system outside of the brain and spinal cord), damaged or severed nerve cells may have some natural regeneration. The nerve fibers grow across the injured area and extend through to their end target (e.g., skin, muscle, etc.). If the injured area is larger than a few millimeters, however, the nerve cells may not regenerate on its own and, if left untreated, permanent sensory loss and paralysis may ensue.

In the peripheral nervous system, a common treatment to repair damaged nerves involves a surgical procedure to harvest a healthy nerve from another part of the patient's body and graft the harvested nerve to bridge the damaged section. Although surgery can successfully repair damaged nerve cells in many cases, these procedures may have several disadvantages. For instance, in most cases, several invasive surgical procedures are required to find suitable donor nerves. Further, damage to nerves at the donor site is quite common, potentially leading to weakening of donor nerves at the expense of the recipient nerves.

Some alternatives to surgical repair of damaged nerves have been developed. These systems typically involve surrounding damaged nerves in a sheath and administering therapeutic drugs or electromagnetic energy to the damaged nerve site. The administration of the therapeutic drugs and/or electromagnetic energy may facilitate nerve regeneration, while the sheath guides the nerve to grow in a desired direction.

Although these systems provide promising alternatives to nerve grafting procedures, they may have several disadvantages. For example, many conventional nerve regeneration systems have limited data processing capabilities. Also, they do not include integrated devices that can deliver therapeutic agents (e.g., drugs, electromagnetic energy, etc.) and monitor biological or chemical responses to the delivered therapeutic agents. Instead, regeneration and growth of damaged nerves may require subsequent exploratory operations, which may be time consuming, costly, and invasive for the patient.

Options for repairing nerves in the central nervous system are much more limited. Currently, the only widely available treatment is to administer therapeutic drugs to the damaged nerves. Drug treatment for spinal injuries has had very limited success. Some developing treatments involve the use of stem cells and the application of simple electric fields, but these treatments have rendered few determinative results thus far.

Thus, there is a need for an improved nerve regeneration system that may overcome one or more of the problems discussed above. In particular, there is a need for an improved nerve regeneration system that can efficiently optimize the treatment parameters, without requiring invasive exploratory techniques.

### SUMMARY

Therefore, various exemplary embodiments of the invention may provide a nerve regeneration system that may include an interactive diagnostic device configured to measure nerve growth, re-growth, and/or connections between severed or otherwise damaged nerve segments.

To attain the advantages and in accordance with the purpose of the invention, as embodied and broadly described herein, one exemplary aspect of the invention may provide a method for treating a body. The method may comprise implanting an elongated lead within a patient's body, the elongated lead having a plurality of electrodes. The plurality of electrodes may be configured to deliver electric stimulation to an area of the patient's body. The method may also include selecting at least one transmitting electrode from among the plurality of electrodes and causing the at least one transmitting electrode to transmit an electric signal to one or more other electrodes to stimulate a damaged nerve.

In accordance with yet another aspect, the present disclosure is directed toward a nerve regeneration system. The system may include an elongated lead configured to be implanted within a patient's body. The system may also include a plurality of electrodes disposed along the elongated lead and configured to deliver electric stimulation to an area of a patient's body. The plurality of electrodes may comprise at least one transmitting electrode in communication with the controller, wherein the at least one transmitting electrode is configured to transmit an electric signal to one or more other electrodes. The controller may be configured to control operation of the at least one transmitting electrode.

According to another aspect, the present disclosure is directed toward a method for treating a body comprising implanting a first elongated lead in a patient's body, the first elongated lead having a first electrode and implanting a second elongated lead within a patient's body, the second elongated lead having a second electrode. The method may also include sequentially energizing the first and second electrodes to create an oscillating electromagnetic field between the electrodes.

In accordance with yet another aspect, the present disclosure is directed toward a system used for a nerve regeneration treatment comprising a first elongated lead configured to be implanted within a patient's body and having a first electrode, and a second elongated lead configured to be implanted within the patient's body and having a second electrode. The system may also include a controller configured to sequentially energize the first and second electrodes to create an oscillating electromagnetic field between the electrodes.

According to yet another aspect, the present disclosure is directed toward a nerve generation system, comprising a controller housing, an elongated lead extending from the housing, at least a portion of the elongated lead being configured to be implanted within a patient's body. The system may also include an anchoring device located at a distal end of the elongated lead, the anchoring device being configured to secure the distal end of the elongated lead to a portion of the patient's body.

According to yet another aspect, the present disclosure is directed toward a nerve generation system having a controller housing, an elongated lead movably coupled to the housing, at least a portion of the elongated lead being configured to be implanted within a patient's body. The system may also include at least one of an electrode and a transducer coupled to the elongated lead, wherein the controller housing comprises a driver assembly configured to move the elongated lead relative to the controller housing.

According to yet another aspect, the present disclosure is directed toward a nerve generation system comprising an elongated tubular member configured to be implanted within a patient's body proximate a damaged nerve and configured to guide growth of the damaged nerve substantially therethrough. The system may include a plurality of electrodes disposed along a length of the tubular member. Each of the electrodes may be configured to deliver an electric stimulation to a portion of the damaged nerve and monitor a response to the applied electric stimulation.

In accordance with still another aspect, the present disclosure is directed toward a tissue manipulating system comprising a sealed housing configured to be at least partially implanted within a body proximate a damaged nerve. The system may also include a fluid port in the sealed housing for receiving fluid. The system may further include an inflatable member in fluid communication with the fluid port. The system may also include a controller configured to control flow of the fluid into and out of the inflatable member, thereby controlling inflation and deflation of the inflatable member.

According to yet another aspect, the present disclosure is directed toward a method for treating a body comprising implanting a housing proximate a damaged nerve. The housing may include at least one advanceable member at least partially disposed therein. The method may also include sequentially actuating the at least one advanceable member to stimulate the damaged nerve tissue and monitoring the damaged nerve's response to the stimulation.

In accordance with still another aspect, the present disclosure is directed toward a method for treating a body comprising depositing a magnetic therapeutic device proximate damaged nerve tissue, the magnetic therapeutic device comprising at least one electromagnet. The method may also include energizing the at least one electromagnet to create a stimulating magnetic field and directing at least a portion of the magnetic field toward the damaged nerve tissue. The method may further include monitoring the damaged nerve's response to the magnetic field.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate various embodiments of the present invention, and, together with the description, serve to explain the principles of the invention.

FIG. 1a illustrates a perspective view of an exemplary embodiment of a nerve regeneration system consistent with the present invention, wherein a fully implanted, multiple-lead nerve regeneration device communicates with an interrogator device.

FIG. 1b provides a schematic diagram illustrating various functional elements of the nerve regeneration system of FIG. 1a.

FIG. 2 illustrates a perspective view of an exemplary embodiment of a nerve regeneration device consistent with the present invention, wherein a nerve regenerator includes a single lead with multiple electrodes.

FIG. 3 illustrates a perspective view of an exemplary embodiment of a nerve regeneration device consistent with the present invention, wherein a nerve regenerator includes a first lead that transmits energy to both a second lead and a third lead.

FIG. 4 illustrates a perspective view of an exemplary embodiment of a nerve regeneration device consistent with the present invention, wherein the nerve regeneration device includes a lead that has a bone screw on its distal end.

FIG. 5a illustrates a side view of an exemplary embodiment of a lead for a nerve regeneration device consistent with the present invention, wherein the lead includes a proximal end configured to be cut to size by an operator.

FIG. 5b illustrates a side view of the lead of FIG. 5a after the proximal end has been cut to size and an internal conductor has been exposed.

FIG. 5c illustrates the lead of FIG. 5b after having been attached to a nerve regeneration device consistent with the present invention.

FIG. 6 illustrates a side view of an exemplary side view of a nerve regeneration device consistent with the present invention, wherein a lead includes a portion that can be advanced or retracted after implantation of the device.

FIG. 7 illustrates an exemplary embodiment of a nerve regeneration system that is implemented using wireless electrode components consistent with the present invention.

FIG. 8 illustrates a perspective view of an exemplary embodiment of a microelectrode array consistent with the present invention.

FIG. 9a illustrates a side view of an exemplary structure for promoting nerve growth associated with nerve regeneration system.

FIG. 9b illustrates an end view of the structure of FIG. 9a.

FIG. 9c illustrates a perspective view of the structure of FIG. 9a.

FIG. 10a illustrates a side view of an exemplary tissue manipulating device that includes an expandable member configured to deliver physical stimulation to nerves, consistent with the present invention.

FIG. 10b illustrates a side view of another exemplary tissue manipulating device that includes retractable projecting elements configured to deliver physical stimulation to nerves, consistent with the present invention.

FIG. 11 illustrates a perspective view of an exemplary embodiment of a magnetic therapeutic device consistent with the present invention.

FIG. 12 illustrates an exemplary application of a nerve regeneration device consistent with the present invention.

### DETAILED DESCRIPTION

Reference will now be made in detail to exemplary embodiments consistent with the invention, examples of which are illustrated in the accompanying drawings. Wherever possible, the same reference numbers will be used throughout the drawings to refer to the same or like parts.

The embodiments described herein are directed toward systems and methods for reconnecting diseased, severed, or otherwise damaged nerves. More specifically, the present embodiments provide a system for causing severed or damaged nerve axons to grow and re-attach to other healthy nerves. Accordingly, the nerve regeneration treatments described herein are directed toward restoring signal transmission capabilities of central and peripheral nervous systems to restore motor control and sensory functions of damaged nerves in patients.

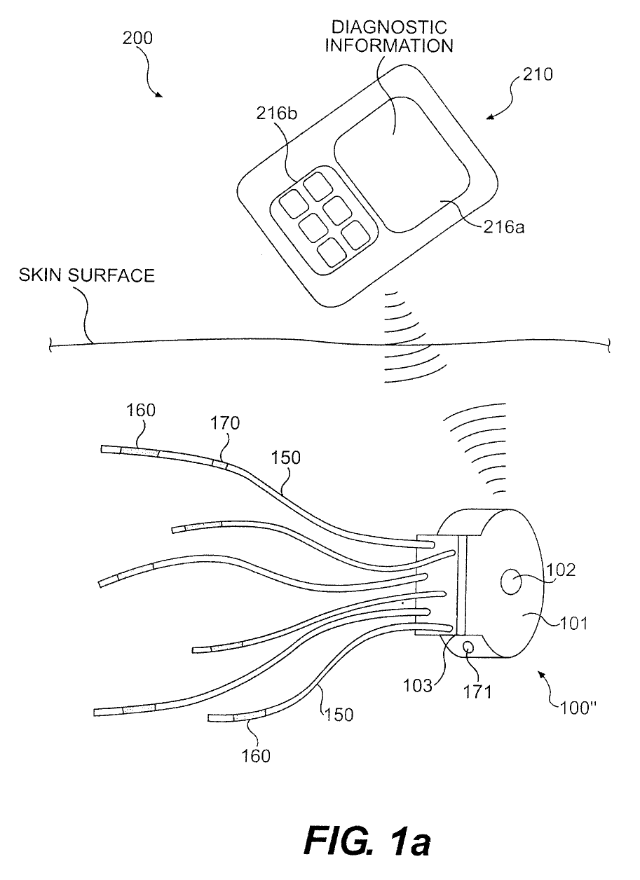

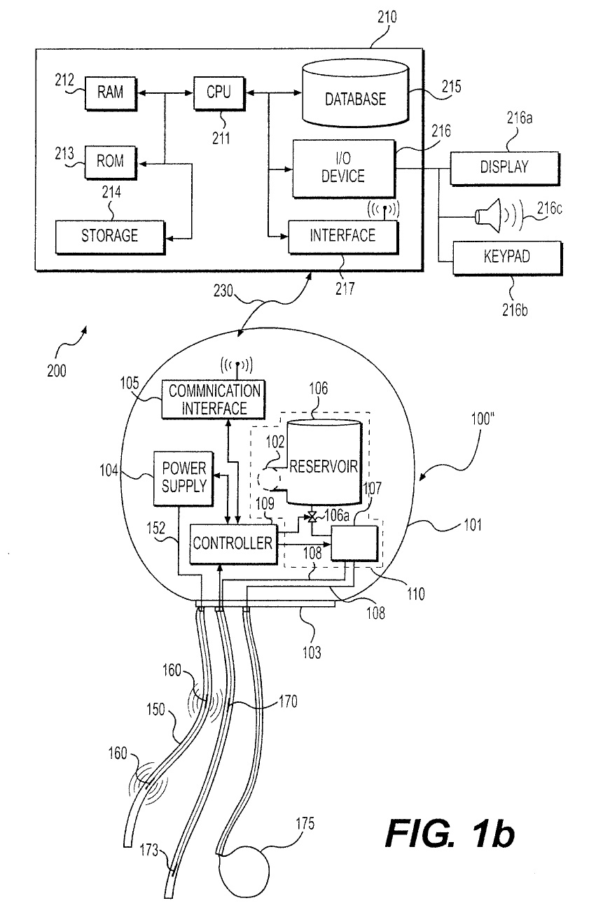

FIG. 1a illustrates an exemplary nerve regeneration system 200 consistent with the disclosed embodiments. Nerve regeneration system 200 may include one or more components that cooperate to regenerate nerves that have been diseased, damaged and/or severed. According to one embodiment, nerve regeneration system 200 may include a nerve regenerator 100″ for implantation in the body of patient at or near damaged nerve cells. Nerve regeneration system 200 may also include an interrogator 210 communicatively coupled to nerve regenerator 100″ and configured to communicate nerve treatment data with nerve regenerator 100″. Nerve treatment data may include, but not be limited to, control signals, diagnostic information, and other information associated with the administration of nerve regeneration treatments.

As illustrated in FIG. 1a, nerve regeneration system 200 may be configured to administer one or more nerve regeneration treatments, monitor nerve regeneration characteristics (e.g., biological, physiological, chemical, and/or electrical signals) in response to the administered treatment, and adjust one or more operational parameters of the nerve regeneration treatment based on the monitored characteristics. According to one embodiment, nerve regeneration system 200 may be configured to operate as an automated treatment and diagnostic system, whereby one or more parameters of nerve regeneration treatment are automatically adjusted, without requiring an external operator's intervention.

Alternatively or additionally, nerve regeneration system 200 may be operated in a “manual” mode. For example, nerve regenerator 100″ may be configured to administer a nerve regeneration treatment based on a control signal provided by a lab technician, doctor, nurse, or other authorized person via an external system (e.g., interrogator 210). During the administration of the treatment, nerve regenerator 100″ may collect patient data, such as nerve regeneration rate, nerve growth, data indicative of nerve response to various stimuli, etc. Nerve regenerator 100″ may provide these data to an external diagnostic system (e.g., interrogator 210) for analysis. Based on the analysis, a lab technician, doctor, nurse, or other authorized person may modify one or more treatment control parameters (e.g., stored in interrogator 210). Interrogator 210 then may subsequently transmit the updated control parameters to nerve regenerator 100″ via a wireless or direct data link. This diagnostic analysis and control cycle may continue during one or more treatment sessions until a desired nerve regeneration result is achieved.

Nerve regenerator 100″ may include a control module 101 that includes a plurality of electrical, mechanical, and/or electromechanical components for aiding in the administration, monitoring, and adaptation of one or more nerve regeneration therapies to damaged nerves. Control module 101 may include a fluid-tight housing having a fluid port 102 for receiving fluid (e.g., therapeutic drugs, air or other fluid for inflating lumens or other securing devices, etc.) for delivery to the patient's body. Control module 101 may also include one or more functional elements 171, such as transducers and/or sensors for monitoring one or more biological, physiological, chemical, and/or electrical conditions associated with the area surrounding control module 101. The number and type of components listed above are exemplary only and not intended to be limiting. For example, control module 101 may include one or more electrodes disposed within or integrally formed on a housing of control module 101 and/or integrally formed on the exterior of control module 101.

Nerve regenerator 100″ may include a plurality of leads 150 communicatively coupled to control module 101 via a header 103. Leads 150 may be flexible, tubular members that may be strategically placed at or near damaged nerves. Leads 150 may each include a hollow, flexible, insulating jacket constructed of plastic, rubber, silicone, or other flexible material. Leads 150 may provide a protective conduit for passing conductors and fluid delivery tubes to areas associated with damaged nerves. For example, leads 150 may provide a conduit for housing conductors that may be coupled to one or more electrodes 160 disposed along the length of leads 150. Alternatively and/or additionally, leads 150 may provide a conduit for housing fluid delivery tubes that may be coupled to one or more transducers 170 (e.g., a drug or other agent delivery mechanism) disposed along the length of one or more leads 150. Alternatively or additionally, leads 150 may include one or more functional elements along its length, not shown but preferably a transducer such as mechanical, electrical, acoustical and/or other transducer, or a sensor such as a physiologic, biologic, electrical, mechanical, acoustical, light or other sensor.

One or more leads 150 may include distal and proximal ends and may be configured to be percutaneously inserted into the body of the patient. The distal end may be adapted for insertion near damaged nerve tissue, while the proximal end may be adapted for connection with control module 101. For example, the distal end of lead 150 may have a thinner diameter than the proximal portion of the lead. Further, the distal end of the lead may be more flexible, thereby allowing a surgeon to manipulate lead placement within the body.

According to one exemplary embodiment, leads 150 may be configured with multiple distal portions such that multiple leads may be inserted within the body without requiring separate connections to control module 101. For example, leads 150 may include multiple attachment connection points such that one or more leads may be interconnected and/or connected to a single “master” lead. As such, leads 150 may be added or removed prior to, during and/or after the initial implantation. In an exemplary embodiment, after therapy has been completed, a proximal portion of a lead is detached, at a connection point, from a distal portion of that lead, avoiding any need to cut the lead, such as if removal of the distal portion is difficult due to tissue in-growth or other physiologic fixation. Each distal end may include one or more electrodes 160, transducers 170, and/or sensors 173.

Leads 150 may include a biodegradable portion that breaks down or dissolves when left in the body for a period of time. According to one exemplary embodiment, leads 150 may be adapted to dissolve to a predetermined diameter, thereby becoming more flexible after implantation and/or to be easier to remove such as at the end of the therapy.

Leads 150 may be coated with a hydrophilic, hydrophobic, or other suitable coating that allows leads 150 to easily slide in and out of the body during implantation or extraction.

Leads 150 may be placed proximate damaged nerves. For example, leads 150 may be placed in and/or around the spinal cord of a patient with a spinal cord injury. Accordingly, the leads may be placed proximate damaged nerves of the central nervous system and may be situated such that a first electrode is on one side of a severed nerve and a second electrode is located on the other side. According to one embodiment, first and second electrodes may be placed equidistant from the damaged area (e.g., vertebral segments above and below spinal cord lesion).

Leads 150 may include one or more integrally formed pockets or loops (not shown) for promoting tissue growth along the length of the lead. Alternatively or additionally, a tissue in-growth cuff, such as a Dacron cuff, may be included along the length of lead 150. According to one exemplary embodiment, these pockets of loops may be coated with therapeutic fluids (e.g., nerve growth agents, stem cells, drugs, etc.)

Alternatively, leads 150 may include one or more devices that prevent the growth of tissue. For example, leads 150 may include radiation generating devices that prevent or slow tissue growth in the surrounding area. This may be particularly advantageous to prevent undesired tissue growth that may block a nerve regeneration path and/or make lead removal difficult. Alternatively, leads 150 may be coated in and/or configured to deliver medications that limit the growth of tissue.

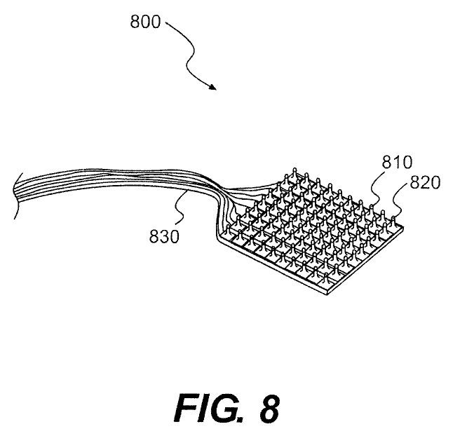

Leads 150 may include an electrode array (such as multi-electrode array 800 of FIG. 8) comprising a plurality of electrodes arranged in a two or three-dimensional array pattern for providing electrode coverage across an area of a patient's body. A first plurality of electrodes may be configured to record single cell neurological activity. In addition, a second plurality of electrodes may be configured to provide stimulation to one or more single cells (such as damaged nerve cells). Alternatively, a plurality of electrodes may be included and configured to record neurological or other cellular activity and provide stimulation or microstimulation to an area of tissue. In an exemplary embodiment, leads 150 include an electrode array (such as the multi-electrode array 800 of FIG. 8) comprising a plurality of electrodes arranged in a two or three-dimensional array pattern for providing information relative to the nerve regeneration, such as to improve therapeutic benefit (e.g. in a closed loop system).

As illustrated in FIG. 1a, nerve regenerator 100″ may be configured to be implanted within the body of a patient via a surgical procedure. Although nerve regenerator 100″ is illustrated as being completely implanted beneath the skin of a patient, it is contemplated that a portion of nerve regenerator 100″ may be located external to the body and/or at the surface of the skin. In one exemplary embodiment, control module 101 may be located at or near the surface of the skin, enabling easy access (e.g. via a syringe and needle) to fluid port 102 for delivering fluids to the control module 101. Regardless of whether nerve regenerator 100″ is implanted completely or partially within the body of the patient, leads 150 may be implanted and situated within the body of the patient at or near damaged nerves, thereby ensuring effective administration of nerve regeneration treatment to the damaged nerves.

Interrogator 210 may be communicatively coupled to nerve regenerator 100″ and configured to communicate information related to nerve regeneration treatment with nerve regenerator 100″. Interrogator 210 may also be configured to analyze treatment information, display treatment information to a patient, health care provider, and/or lab technician, and provide treatment recommendations based on the analyzed treatment information.

Interrogator 210 may include any type of diagnostic tool or computer system that may be adapted to communicate with nerve regenerator 100″. Interrogator 210 may include, for example, a handheld diagnostic tool, a personal desktop assistance (PDA), a wireless telephone or other communication device, a handheld computer gaming device, a desktop or notebook computer system, or any other processor-based device that is configured to execute diagnostic and/or control software associated with nerve regeneration system 200, receive data input from the user, and/or output data to the user via an interface. For example, as illustrated in FIG. 1a, interrogator 210 may embody a handheld communication device that includes a screen 216a for displaying diagnostic information to a user, a keypad 216b for receiving commands from the user, and one or more communication devices for wirelessly communicating data with nerve regenerator 100″. Although FIG. 1a illustrates interrogator 210 as being in wireless communication with nerve regenerator 100″, it is contemplated that interrogator 210 may communicate data to nerve regenerator 100″ via a wireline connection or direct data link (e.g., serial, parallel, USB, etc.). As such, interrogator 210 and nerve regenerator 100″ may each include data ports that support wire-based communication protocols.

According to an exemplary embodiment and as will be described in greater detail below, the presently disclosed nerve regeneration systems and associated methods involve passing electric current from at least one electrode to one or more other electrodes, providing a therapeutic electrical field therebetween. The field created between the electrodes may be an oscillating field generated by alternately applying positive and negative pulses of DC current between the electrodes. For example, a first electrode transmits DC current to a second electrode for a predetermined first time period to promote nerve growth in one direction. Subsequently, the polarity of the current is switched and the second electrode transmits the DC current to the first electrode to promote nerve growth in another direction. The DC current may be set at a predetermined level, such as between 200-1000 microamps (or other appropriate level). In an alternative embodiment, the DC current may vary during each pulse.

According to one embodiment, the duration of the pulses are established to be less than an axon “die back” period (i.e., the amount of time that an oppositely facing axon can withstand electric energy before beginning to degenerate). Die back periods have been estimated through experimentation to begin at time periods greater than one hour. According to another embodiment, the duration of the pulses are established to be at least 30 seconds such as to be long enough to cause axonal growth, as also has been estimated through experimentation.

In addition to reducing the die back in nerve axons, oscillating fields have been shown to reduce electrolysis and other toxin-producing nerve reactions that may be associated with electromagnetic fields. Furthermore, prolonged electric field exposure may, in some cases, adversely interfere with the effect of drugs and other types of nerve regenerative treatments. Accordingly, it may be advantageous to set pulse durations sufficiently long to promote nerve growth, while, at the same time, keeping the durations short enough to limit adverse effects associated with prolonged constant DC electric fields. According to one exemplary embodiment, pulse durations may initially be established at approximately thirty (30) seconds. This duration may be adjusted (e.g. increased) in accordance with the diagnostic methods, which are described in greater detail below.

FIG. 1b provides a schematic illustration of certain components and features associated with an exemplary nerve regeneration system 200 consistent with the disclosed embodiments. Specifically, FIG. 1b illustrates certain internal components associated with nerve regeneration system 200 and its constituent components and subsystems.

Control module 101 may include a housing that may be sealed to protect one or more components disposed inside the housing from the surrounding environment. Control module 101 may be made of a lightweight plastic, metallic (e.g., titanium), or composite material. According to one embodiment, control module 101 may be secured to a portion of the patient's body (e.g., skin, tissue, bone, etc.) using sutures, screws, or any other suitable device for fastening control module 101 to the patient's body. In embodiments where control module 101 is located outside of the patient's body, control module 101 may be secured onto the body using a strap or band.

Control module 101 may include a removable header 103 that provides an interface for passing electrical conductors or fluid delivery tubes through the wall of the housing of control module 101. Header 103 may be slidably coupled to a portion of the housing of control module 101. Alternatively, header 103 may be secured to the housing such as such as via screws or a welded joint.

Header 103 may include one or more interfaces for connecting leads 150. For example, header 103 may include a female, nut-type connector that may mate with a male, bolt-type connector associated with lead 150 to form a passage through header 103 for passing conductors and fluid delivery tubes therethrough. Header 103 may include any number of connection interfaces, providing access for several different leads. When not in use, the connection interfaces may be covered and/or sealed to protect control module 101 and any of its components from the surrounding environment.

In some exemplary embodiments, control module 101 may be configured to deliver electrical, magnetic, light energy, chemical stimulants and/or other substances such as stem cells, to damaged nerve cells. For instance, as shown in FIG. 1b, control module 101 may include a power supply 104 configured to provide power to one or more components of control module 101; a communication interface 105 for transmitting patient data to and receiving control signals and configuration data from an external system (e.g., interrogator 210); a fluid delivery system that includes a reservoir 106 for storing fluid to be delivered to the patient's body and a fluid delivery device 107 for delivering fluid to the patient via one or more fluid delivery tubes 108; and a controller 109 for collecting, analyzing, controlling, monitoring, and/or storing information associated with the operation of control module 101.

Power supply 104 may include a battery, a fuel cell, a charge storing device, a transformer, a signal generator, an AC or DC power source, and/or any other device for providing power to operate control module 101. According to one embodiment, power supply 104 may include a rechargeable battery that may be inductively coupled to an external battery charger for wirelessly charging the power supply. In some cases, power supply 104 may be electrically coupled to an external power source via a power cable.

Power supply 104 may be communicatively coupled to one or more electrodes 160 via conductors 152. Electrodes 160 may embody high-conductivity metallic or metallic alloy materials such as platinum and/or platinum-iridium metals and may be adapted to deliver electrical energy to damaged nerves and/or tissue associated therewith. Electrodes 160 may also be configured to monitor electrical signals and other patient data, such as during energy delivery and/or at a time when energy delivery has ceased. Electrodes 160 may be routed through lead 150 and, accordingly, may be strategically implanted at or near the damaged nerve sites.

According to one embodiment, electrodes 160 may be selectively configured as stimulation devices and sensing devices. For example, electrodes 160 may be coupled to a multiplexer that, when operated by controller 109, may be configured to toggle electrodes between “transmit” and “sense” modes.

Electrodes 160 may also include one or more micro-electrodes (not shown) protruding along the length of electrode 160. According to one embodiment, these micro-electrodes may include fibrous conductive materials (e.g., nanofibers, etc.) for enhancing the energy delivery capabilities associated with each electrode 160.

According to one embodiment, electrodes 160 may vary in length (e.g., from about 0.5 to 5 millimeters) and may have a relatively small diameter (e.g., a diameter of less than a human hair). As such, electrodes 160 may be small enough to be implanted in the spinal column and/or portions of the brain for delivering electro-therapeutic stimulants to portions of the central nervous system.

Communication interface 105 may include a communication module adapted to transfer information between control module 101 and an external diagnostic system, such as interrogator 210. Communication interface 105 may include an antenna to support wireless communication and/or a communication port to support direct connection to one or more external systems. In an exemplary embodiment, communication interface 105 may be adapted to support multiple wireless communication protocols such as, for example, Bluetooth, WLAN, cellular, other RF, and/or microwave communication formats. Alternatively or additionally, communication interface 105 may be adapted to support wire-based communication platforms and media such as, for example, serial (USB), parallel, Firewire, Ethernet, and optical communication platform or medium.

Fluid delivery system 110 may include one or more components for enabling fluid flow associated with nerve regeneration system 200. Fluid delivery system 110 may be configured to dispense therapeutic drugs or other agents (e.g., pain killers, nerve growth agent, proteins and fluids for promoting healthy nerve growth environment, stem cells, etc.) to the patient's body. Fluid delivery system 110 may also be configured to deliver fluids for inflating one or more balloons adapted to secure leads 150 and/or control module 101 in a particular location.

As mentioned above, fluid delivery system 110 may include reservoir 106 in fluid communication with fluid port 102 and fluid delivery device 107 configured to deliver fluid stored in reservoir 106 to one or more transducers 170 via one or more fluid delivery tubes 108. Fluid port 102 may enable delivery of fluids to the control module 101, without requiring removal or disassembly of the control module 101. In some exemplary embodiments, fluid port 102 may include a re-sealable membrane, such as, for example, a silicone septum similar to those used in implantable infusion pumps, adapted to re-seal after a puncture by a hypodermic or other anti-coring needle. Alternatively, fluid port 102 may include a mechanical valve percutaneously accessible by a needle or other flow conduit. Although FIG. 1 is illustrated as having a single fluid port 102, additional fluid ports and/or fluid delivery mechanisms may be provided. For example, if multiple therapeutic drugs are required as part of a nerve regeneration treatment, the fluid delivery system 110 may include multiple fluid ports 102 and/or multiple fluid delivery mechanisms to allow separate injection and/or handling of the drugs or other agents (e.g. stem cells) in the system.

Reservoir 106 may be in fluid communication with fluid port 102 and configured to store the fluid delivered to fluid port 102. Reservoir 106 may embody a fluidly isolated compartment for storing a supply of fluids for use by fluid delivery system 110. Although control module 101 is illustrated as having a single reservoir, additional reservoirs 106 may be provided. For example, in an exemplary embodiment, the fluid delivery system may include at least a first reservoir and a second reservoir. The first reservoir may contain nerve growth agent, while a second reservoir may contain a photoreactive, luminescent and/or radiolabeled dye that, when injected into the body and exposed to a detection device such as a phototransmitter and camera/receiver or a radiographic detector such as a fluoroscope, may aid in observing nerve activity and/or nerve regenerative growth during and/or after therapeutic treatments.

Fluid delivery device 107 may control the fluid flow associated with nerve regenerator 100″. According to one embodiment, fluid delivery device 107 may include a pump operatively coupled to controller 109 and adapted to operate in response to command signals received from controller 109. Fluid delivery device 107 may be coupled to reservoir 106 via a valve 106a, which may be operated by controller 109 to enable fluid flow from reservoir 106 to fluid delivery device 107. When multiple reservoirs 106 are used, a group of reservoirs may be selectively coupled to fluid delivery device 107 via a single controller-operated valve. Accordingly, by selectively coupling one or more reservoirs 106 to fluid delivery device 107 using valves (e.g., valve 106a) on an ad hoc basis, a single delivery device may be used to dispense multiple fluids required by nerve regeneration system 100″, reducing costs and implant size typically needed for multiple fluid delivery devices.

Fluid delivery device 107 may be fluidly coupled to one or more fluid delivery tubes 108, which may be routed through leads 150. When nerve regenerator 100″ is implanted, fluid delivery tubes 108 and/or leads 150 may be placed in desired locations proximate the damaged nerves. Fluid delivery tubes 108 may be terminated in one or more needles or other flow conduits that protrude from lead 150 for depositing fluid (e.g., therapeutic drugs) to damaged nerve sites. Alternatively or additionally, fluid delivery tubes 108 and/or leads 150 may include openings, or a porous material to release fluid into the damaged nerve sites. Alternatively or additionally, an electromagnetic field may be generated to deliver drugs or other agents via iontophoresis. Alternatively or additionally, fluid delivery tubes 108 may be used to deliver stem cells to the damaged nerve sites.

In addition to dispensing therapeutic drugs, fluid delivery system 110 may be used to secure nerve regenerator 100″ and/or one or more leads 150 in the desired location. For example, in an exemplary embodiment, fluid delivery system 110 may include one or more inflatable balloons 175 attached to the end of fluid delivery tube 108, which may be coupled to the fluid delivery device 107. When fluid is delivered to balloon 175, balloon 175 inflates, thereby securing leads 150 in place. These balloons may substantially prevent nerve regenerator 100″ and/or one or more leads 150 from excessive movement in the body.

As explained, the fluid delivery system 110 may include a separate reservoir 106 containing a filler agent (e.g., air, saline, etc.) and fluid delivery device 107 delivers the filler agent to inflatable balloons 175. Alternatively or additionally, fluid delivery device 107 may also be adapted to dispense materials that aid in determining the effectiveness of nerve regeneration treatments. For example, fluid delivery device 107 may dispense light sensitive fluids or dyes that, when exposed to light or suitable electromagnetic radiation (e.g., generated by an LED, optical, RF, or microwave generator associated with one or more leads 150), may aid in detecting nerve endings. Alternatively or additionally, fluid delivery device 107 may dispense a radiolabeled isotope or other radiographic material that, when imaged by a fluoroscope, may aid in visualizing nerves and/or nerve growth. By measuring axon (e.g., nerve ending) locations periodically, a growth rate of the nerve endings may be determined.

Controller 109 may include any type of microcontroller or processor-based device that may be configured to control one or more operational aspects of nerve regenerator 100″. According to one exemplary embodiment, controller 109 may be operated manually or automatically. For example, in a manual operating mode, controller 109 may be configured to receive commands from an external device (e.g., interrogator 210) for operating nerve regenerator 100″ via communication interface 105. Alternatively, in an automated mode, controller 109 may be configured to control the operations of nerve regenerator 100″ without requiring separate commands from the external device. In either case, controller 109 may be adapted to store and/or transmit operation data associated with nerve regenerator 100″, treatment data associated with a patient, and other information related to nerve regeneration treatments for later analysis by interrogator 210 or other suitable diagnostic device.

Controller 109 may be electrically coupled to power supply 104 and configured to regulate power output to components associated with nerve regenerator 100″. Additionally, controller 109 may include electronic switching and logic circuitry for operating power supply 104 to provide electromagnetic stimulation via electrodes 160 to damaged nerves. According to one embodiment, controller 109 may be adapted to control the voltage and/or current levels provided by power supply 104. In addition, controller 109 may be configured to control the frequency of the electromagnetic stimulation generated by power supply 104. According to another embodiment, controller 109 may include a multiplexer for selectively coupling one or more electrodes to power supply 104. As such, controller 109 may be configured to select one or more electrodes from a plurality of electrodes that receive electric energy from power supply 104.

Controller 109 may also be configured to control an oscillating electromagnetic field (e.g. a switching DC field, such as a constant current DC field created by flowing approximately 200-1000 microamps from a first electrode, through tissue, to a second electrode) for stimulating nerve regeneration. As explained, controller 109 may be electrically coupled to power supply 104, which may include a signal generator for generating an electromagnetic field. According to one embodiment, controller 109 may be configured to control the frequency, period, and amplitude of the oscillating electromagnetic field so as to minimize degeneration of anodally facing axons and to stimulate growth of cathodally facing axons. Accordingly, the electromagnetic field generated by power supply 104 may be adjusted by controller 109 so as to maximize the growth rate of nerves facing a first direction, without desensitizing or damaging nerves facing a different direction (e.g. an opposite direction).

Controller 109 may also be electrically coupled to fluid delivery device 107 to control the delivery of fluids associated with nerve regenerator 100″. For example, controller 109 may be configured to provide control signals for operating reservoir selecting valves 106a. Alternatively or additionally, controller 109 may be configured to operate fluid delivery device 107 to deliver therapeutic drugs to damaged nerves and/or to inflate/deflate balloon 175. Controller 109 may be configured to operate one or more transducers 170. Transducer 170 may include, for example, a fluid delivery mechanism such as a micropump (e.g. a MEMS fluid delivery mechanism) or a micro-syringe or plunger for regulating an amount of fluid delivered to a damaged nerve. Transducer 170 may also include one or more of: drug delivery elements; drug storage depots; audible transducers (e.g. for alarm and alert conditions); magnetic field generators; heat generators; cooling generators; electrodes; fluid delivery pumps; iontophoresis elements; powder delivery mechanisms; vibration generating mechanisms; and combinations thereof. According to another embodiment, transducer 170 may include a device for depositing tagging agents or other materials for monitoring nerve parameters. Tagging agents may include photosensitive materials, dying agents such as radiolabeled agents, RFID devices, or other types of devices that may be used to monitor a nerve parameter. Alternatively or additionally, transducers 170 may include one or more devices for emitting wave radiation such as, for example, an LED, a fluorescent light, a microwave generating device, or an infrared generator. These radiation emitting devices may be used for nerve treatment or, alternatively, may be operated to react with a tagging agent to measure a nerve parameter and/or a change in a nerve parameter. According to still another embodiment, transducers 170 may include heating or cooling elements that, when operated by controller 109, may emit temperature stimulation. It is contemplated that one or more transducer 170 may be included as part of nerve regenerator 100″, integral to one or more components of nerve regenerator 100″ or included as a standalone component of nerve regenerator 100″.

Controller 109 may be in data communication with one or more sensors 173 and may be configured to receive/collect information associated with nerve treatment, including biological, physiological, chemical, and/or electrical data associated with the patient. Sensors 173 may include, for example, mechanical sensors, electrical sensors, magnetic sensors, acoustic sensors, light sensors, radiation sensors, chemical sensors, physiological sensors, temperature sensors, voltage sensors, current sensors, blood sensors, glucose sensors, pH sensors, EKG sensors, EEG sensors, single cell sensors such as arrays of microelectrodes configured to detect single cell neuron action potentials, LFP sensors, ECoG sensors, EMG sensors, and/or any other type of sensors adapted to collect data associated with a patient response (e.g., a patient physiological response) to nerve regeneration treatment. Patient response, as the term is used herein, may include, but is not limited to: a cellular (nerve) growth measurement; a hormonal reaction or change; a release of toxin or other chemical or agent; a physiologic reaction parameter; an EEG parameter; an EKG parameter; an EMG parameter; a parameter measured by implanted sensor; a parameter measured by external sensor; a parameter measured by completing a patient questionnaire; a parameter measured by touching the patient; parameter measured by asking the patient to move a portion of his/her body; a parameter measured after injecting an agent such as a radiolabeled or luminescent cellular tagging agent; a parameter which is a surrogate of another parameter; a pin-pick test parameter; a light-touch parameter; a motor function parameter; an evoked potential parameter; or any other parameter indicative of a patient response to nerve treatment. Data received by sensors 173 may be collected in controller 109 and provided to interrogator 210 through communication interface 105 via communication link 230.

Communication link 230 may include any network or data link that provides two-way communication between nerve regenerator 100″ and an external diagnostic system, such as interrogator 210. For example, communication link 230 may communicatively couple nerve regenerator 100″ to interrogator 210 across a wireless networking platform such as, for example, a cellular. Bluetooth, microwave, point-to-point wireless, point-to-multipoint wireless, multipoint-to-multipoint wireless, or any other appropriate communication platform for networking a number of components. Although communication link 230 is illustrated as a wireless communication link, communication link 230 may include wireline links such as, for example, serial, parallel, USB, fiber optic, waveguide, or any other type of wired communication medium.

As explained, interrogator 210 may be a processor-based system on which processes and methods consistent with the disclosed embodiments may be implemented. For example, as illustrated in FIG. 1b, interrogator 210 may include one or more hardware and/or software components configured to execute computer programs. The computer programs may include, for example, diagnostic software for analyzing nerve regeneration treatments, evaluating the effectiveness of the treatments, modifying one or more parameters of the treatments, and/or controlling operation of nerve regenerator 100″.

For example, interrogator 210 may include one or more hardware components such as, for example, a central processing unit (CPU) 211, a random access memory (RAM) module 212, a read-only memory (ROM) module 213, a storage 214, a database 215, one or more input/output (I/O) devices 216, and an interface 217. Alternatively or additionally, interrogator 210 may include one or more software components such as, for example, a computer-readable medium including computer-executable instructions for performing methods consistent with certain disclosed embodiments. It is contemplated that one or more of the hardware components listed above may be implemented using software. For example, storage 214 may include a software partition associated with one or more other hardware components of interrogator 210. Interrogator 210 may include additional, fewer, and/or different components than those listed above. It is understood that the components listed above are exemplary only and not intended to be limiting.

CPU 211 may include one or more processors, each configured to execute instructions and process data to perform one or more functions associated with interrogator 210. As illustrated in FIG. 1b, CPU 211 may be communicatively coupled to RAM 212, ROM 213, storage 214, database 215, I/O devices 216, and interface 217. CPU 211 may be configured to execute sequences of computer program instructions to perform various processes, which will be described in detail below. The computer program instructions may be loaded into RAM for execution by CPU 211.

RAM 212 and ROM 213 may each include one or more devices for storing information associated with an operation of interrogator 210 and/or CPU 211. For example, ROM 213 may include a memory device configured to access and store information associated with interrogator 210, including information for identifying, initializing, and monitoring the operation of one or more components and subsystems of interrogator 210. RAM 212 may include a memory device for storing data associated with one or more operations of CPU 211. For example, ROM 213 may load instructions into RAM 212 for execution by CPU 211.

Storage 214 may include any type of mass storage device configured to store information necessary for CPU 211 to perform processes. For example, storage 214 may include one or more magnetic and/or optical disk devices, such as hard drives, CD-ROMs, DVD-ROMs, or any other type of mass media device.

Database 215 may include one or more software and/or hardware components that cooperate to store, organize, sort, filter, and/or arrange data used by interrogator 210 and/or CPU 211. For example, database 215 may include historical treatment settings (e.g., drug dosages, drug delivery schedules, electromagnetic treatment schedules, electromagnetic treatment power settings, etc.), nerve regeneration data (e.g., nerve growth rate, etc.), patient treatment response data (e.g., EKG data, EEG data, etc.), and/or any other type of data that may be used to diagnose and/or control nerve regenerator 100″. CPU 211 may access the information stored in database 215 for comparing the current treatment levels (and patient responses associated therewith) with historical treatment levels to establish a nerve regeneration treatment. Alternatively or additionally, historical data may be used to customize threshold levels used in the analysis of patient data. Thus, threshold levels for patients that experience greater nerve regeneration may be set higher than threshold levels for patients whose nerve regeneration rate lags behind a normal level, enabling more aggressive treatment options for highly responsive nerves. It is contemplated that database 215 may store additional and/or different information than that listed above.

I/O devices 216 may include one or more components configured to communicate information with a user associated with interrogator 210. For example, I/O devices may include a console with an integrated keypad 216b and/or mouse to allow a user to input parameters associated with interrogator 210. I/O devices 216 may also include a display 216a including a graphical user interface (GUI) for outputting information on a monitor. I/O devices 216 may also include peripheral devices such as, for example, a printer for printing information associated with interrogator 210, a user-accessible disk drive (e.g., a USB port, a floppy, CD-ROM, or DVD-ROM drive, etc.) to allow a user to input data stored on a portable media device, a microphone, a speaker system 216c, or any other suitable type of interface device.

Interface 217 may include one or more components configured to transmit and receive data via a communication network, such as the Internet, a local area network, a workstation peer-to-peer network, a direct link network, a wireless network, or any other suitable communication platform. According to one embodiment, a clinician or other caregiver uploads information and/or downloads commands to interface 217 from a location remote from the patient, such as an information transfer over the Internet. For example, interface 217 may include one or more modulators, demodulators, multiplexers, demultiplexers, network communication devices, wireless devices, antennas, modems, and any other type of device configured to enable data communication via a communication network.

Interrogator 210 may be configured to provide an interface that allows users (e.g., patient, health care provider, etc.) to modify one or more nerve regeneration treatment parameters after implantation of nerve regenerator 100″ into the patient's body. According to one embodiment, information can be transferred at any time to/from interrogator at any time (e.g. during surgery, within 1 hour of implantation, more than 24 hours after implantation and more than 30 days after implantation, etc.) Interrogator 210 may include software that provides users with an interface screen that includes one or more user-adjustable treatment parameters (e.g., drug dosage, drug delivery schedule, electromagnetic treatment schedule, electromagnetic field parameters (e.g., voltage level, electric and magnetic field direction, etc.)). Once established, users may upload the control parameters onto controller 109 associated with control module 101. Accordingly, controller 109 may administer the treatment in accordance with the user-defined parameters. In an alternative embodiment, interrogator 210 or a portion of interrogator 210 is configured to reside at a location remote from the patient, such that a caregiver can transfer commands or other information via wired or wireless communication means, such as the Internet. Interrogator 210 may be configured to communicate information with nerve regenerator 100″ at any time (e.g., during and after surgery, during nerve treatment sessions, etc.). Additionally, interrogator 210 may include a web interface that allows user to communicate with interrogator 210 and/or nerve regenerator 100″ remotely (via the Internet, telephone, etc.). According to an alternative embodiment, interrogator 210 may include a probe, which is configured to pass through the skin to access the control module 101 of nerve regenerator 100″ to transfer power and/or information from interrogator 210 to control module 101.

In some situations, nerve regenerator 100″ and/or one or more devices associated therewith may require periodic configuration and/or calibration to operate properly. Accordingly, interrogator 210 may also be configured to initiate a configuration and/or calibration subroutine for nerve regenerator 100″ and/or its constituent components. For example, should a sensor 173 for measuring electrical signals associated with nerve cells become out of calibration (e.g., as identified by an unrecognizable signal and/or excessive amount of electrical noise in the detected signal), interrogator 210 may be configured to calibrate the electrical sensor by providing a test signal and adjusting a sensor parameter (e.g., gain, etc.) associated with the sensor to cancel or filter any excessive noise. In addition, interrogator 210 may be configured to initiate a reset sequence for restoring one or more parameters associated with nerve regenerator 100″ to a default (e.g., factory/manufacturer preset) condition.

Configuration and/or calibration subroutines may be required to be performed at least once prior to deployment of nerve regenerator 100″ within the body of a patient to ensure proper operation. Additionally, the calibration subroutine may include one or more initial diagnostic tests to gather control data to be used as a benchmark for nerve regenerator treatments.

Nerve regenerator 100″ may include an integral alarm routine that monitors the device parameters or critical health parameters of the patent and provides an audio, visual, or tactile alarm if one or more of the device parameters or health parameters are inconsistent with predetermined levels. According to one embodiment, integral alarm routine is configured to monitor one or more device parameters such as battery power level, nerve stimulation properties (e.g., electric field), and/or lead movement (e.g., vibration, change in resistance, or other parameter that may be indicative of a loose lead). Alarm routine may compare each of these parameters with a predetermined threshold. If a monitored device parameter deviates from the predetermined threshold, alarm routine may operate one or more system alarms. These alarms may include audio, visual, or tactile alarms and may be generated by nerve regenerator 100″ and/or interrogator 210.

Alternatively or additionally, integral alarm routine may be configured to monitor a device performance or therapy outcome parameter. For example, alarm routine may monitor nerve growth, nerve connectivity, or a toxicity measurement associated with damaged nerves (e.g. a toxicity measurement based on a toxicity level or surrogate measured by one or more integral sensors, such sensors including but not limited to: electrodes and other electromagnetic sensors; temperature sensors such as thermocouples; optical sensors; pH sensors; blood sensors; gas sensors such as oxygen or hydrogen sensors; electrolysis or microdialysis sensors; dialysis or microdialysis sensors; etc). The alarm routine may provide one or more alarms for notifying an operator (e.g., clinician, doctor, patient, etc.) that a certain therapy parameter has been met. For example, alarm routine may provide a notification to the operator that a particular nerve growth goal has been achieved. Alternatively, alarm routine may provide a notification to the operator that nerve growth has stagnated for a predetermined time limit. Alternatively or additionally, alarm routine may be adapted to notify an operator if a toxicity level associated with damaged nerve tissue has reached a predetermined limit. The alarm routine may convey alarm information to a location remote from the patient, such as via the internet to a separate health care facility or doctor's office.

It is contemplated that, in addition to providing a notification signal, alarm routine may be configured to take certain preventative measures to correct a condition that caused the alarm. For example, if a toxicity level exceeds a predetermined limit, alarm routine may provide a command signal to controller 109 requesting the delivery of an anti-toxic agent to control the toxicity level.

According to yet another embodiment, alarm routine may be configured to monitor certain patient parameters. For instance, alarm routine may be configured to monitor a temperature (e.g., to detect infection), a pressure, an acceleration (e.g., to detect a fall, seizure, or other undesired patient movement), or any other patient parameter. If a patient parameter exceeds a predetermined (e.g., operator-defined) limit, the alarm may notify an operator.

Alarm routine may be programmed and/or modified by an operator via an external system (e.g., interrogator 210). According to one embodiment, alarm routine may provide a password protected access interface. Accordingly, an operator may program alarm routine via the Internet, telephone, or other communication network using the password protected access.

According to another embodiment, nerve regeneration system 200 may be configured to perform a permission routine. Permission routine may be activated when a system configuration or other parameter is to be initially set or modified in a secured manner. The permission routine may use one or more of: a password; a restricted user logon function; a user ID; an electronic key; a electromechanical key; a mechanical key; a specific Internet IP address; and other means of confirming the identify of one or more operators prior to allowing a secure operation to occur.

Nerve regeneration system 200 may also be configured to perform a clinician confirmation routine. Clinician confirmation routine may be activated prior to the system making a change to a system parameter, such as an energy delivery parameter. A user interface (e.g., such as screen 216a or interrogator 210) may query the clinician if the change is “OK?”—and the system requires a confirmatory response from the clinician prior to implementing the change. The user interface may include a touch screen which includes “YES” and “NO” fields for the clinician to touch. In a preferred embodiment, the clinician has previously entered a security password or other permission routine (e.g. a fingerprint scan) requirement to prevent unauthorized confirmation of system parameter changes.

According to one embodiment, in addition to providing electric stimulation and therapeutic agent delivery, nerve regenerator 100″ may be configured to provide additional types of energy which may enhance nerve regeneration treatments. For example, nerve regenerator 100″ may be configured to provide one or more of: heat, cooling, radiation, light, light activated drugs, ultrasound, magnetic field, stem cell delivery, electrochemical agent delivery, dialysis treatment (e.g., microdialysis), or any other type of treatment. These treatments may be provided by adapting control module 101 and/or leads 150 to include appropriate transducers 170 or other functional elements to provide the desired treatments. For example, one or more leads 105 may be adapted with temperature control elements for providing heating and cooling stimulation. Alternatively, control module 101 may include an ultrasound device for administering ultrasound treatment to surrounding tissue. According to yet another embodiment, control module 101 may include a microdialysis device for administering dialysis treatment.

Nerve regenerator 100″ may include a memory storage component. For example, control module 101 may include a non-volatile RAM or ROM memory device, flash memory device, or any other device for storing data. As such, nerve regenerator 100″ may be configured to store historic functional and/or performance data such as, for example, nerve growth data, alarm data, clinician information (e.g., clinician modification to the system or parameters), or any other type of data.

Memory may be accessible to an external device (e.g., wired or wireless). As such, the external device may be accessible over the Internet, telephone, or other communication network. Accordingly, an operator can remotely download data from and upload data to memory. For instance, an operator can download monitored patient data collected during previous nerve treatment sessions from memory. Alternatively, operator can upload control parameters, alarm threshold levels, software and/or firmware updates for controller, or any other operational parameters to memory. According to one embodiment, memory may be stored in a “ring buffer”, whereby older information is written over as memory becomes full.

Nerve regenerator 100″ may be programmable and/or adjustable by an operator (e.g., clinician, physician, patient, etc.) and configured to allow an operator to modify two or more system parameters. According to one exemplary embodiment, a plurality of nerve regeneration treatment parameters may be modified simultaneously during the nerve regeneration treatments. Alternatively or additionally, one or more nerve regeneration treatment parameters may be modified during the application of another type of nerve regeneration treatment. Nerve regeneration treatment parameters may include, for example, electromagnetic (EM) field strength; EM field direction; EM field pattern; EM field current; EM field voltage; specific elements (e.g. electrodes) receiving energy; pattern of elements receiving energy; type of elements receiving energy; combination of elements receiving energy; duty cycle of energy delivery; frequency of energy delivery; period of energy delivery; off-time of energy delivery; energy type parameter; energy location of delivery parameter; drug delivery parameter; mechanical actuator (e.g. intentional trauma) parameter; magnetic field parameter; light intensity delivered parameter; chemical delivery parameter; radiation delivery parameter; heat energy delivery parameter; position of therapy delivering element; and type of therapy delivering element.

According to one exemplary embodiment, nerve regenerator 100″ and/or controller 109 may be adapted to adjust multiple (e.g., two at a time, three at a time, etc.) nerve regeneration treatment parameters automatically or in response to a user command signal deliver, for example, via interrogator 210. These treatment parameters may be adjusted “on-the-fly”, without requiring shutdown of other nerve regenerative treatment functions.

Treatment parameters may be adjusted based on one or more diagnostic procedures performed by nerve regenerator 100″ or interrogator 210. For example, a clinician may start by applying a first type of nerve regeneration treatment as a “control” treatment. Neurological responses may be measured to determine the damaged nerve's response to the first type of nerve treatment. The clinician may provide a control signal to controller 109 to introduce a second type of nerve regeneration treatment, and observe the damaged nerve's response to the simultaneous treatment. Parameters associated with the first and second nerve regeneration treatments may simultaneous or iteratively be adjusted to determine the effects different interactions of the treatments on damaged nerve.

For example, during the application of an electric stimulation treatment to a damaged nerve, a clinician may send a command signal to controller 109 to activate a heating element of transducer 170 to observe the effects of temperature stimulation coupled with electric stimulation on nerve regeneration. Alternatively, during the application of electric stimulation treatment, a clinician may send a command signal to pulse apply light, microwave, infrared, or other wave radiation to determine the cumulative effects of different types of stimulants on nerve regeneration.

Alternatively or additionally, multiple treatment parameters associated with a single nerve regeneration treatment may be adjusted. For example, during the application of electric nerve stimulation, controller 109 may be configured to adjust a field direction, a field pattern, a field strength, field current, and/or field voltage of the electromagnetic field. Alternatively or additionally, controller 109 may be configured to designate which electrodes are configured to transmit energy and which electrodes are configured to receive energy.

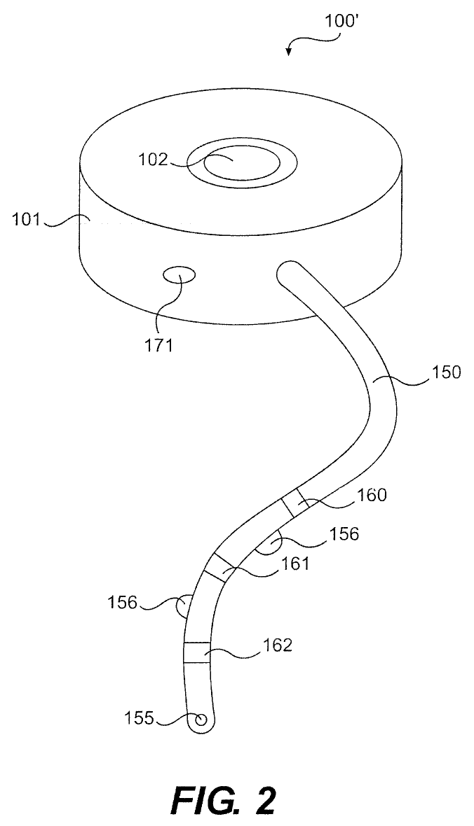

FIG. 2 provides a perspective view of an exemplary nerve regenerator 100′ consistent with the disclosed embodiments. As illustrated in FIG. 2, nerve regenerator 100′ may comprise a single lead 150 that includes a plurality of electrodes 160-162. Each of electrodes 160-162 may be configured to deliver electric stimulation to an area of a patient's body that comprises one or more damaged nerves. According to one embodiment, a DC current (e.g. a current of 200-1000 microamps) is passed between one or more pairs of the electrodes of nerve regenerator 100′. In another preferred embodiment, a constant DC current is applied between any pair of electrodes in a first direction for a period of at least thirty (30) seconds but less than one (1) hour, after which (although not necessarily immediately thereafter), current is applied between that electrode pair in the opposite direction for a period of at least thirty (30) seconds but less than one (1) hour. Additionally, each of electrodes 160-162 may be configured to collect, receive, and/or monitor electrical, chemical, physiological, and/or biological activity associated with the surrounding areas.

Lead 150 may include one or more holes 155 or loops 156 for securing lead 150 in a desired location. For example, upon implantation of nerve regenerator 100′, lead 150 may be located near or around damaged nerves to maximize the treatment capabilities of nerve regenerator 100′. Once arranged, lead 150 may be secured to bone, fascia, ligaments or other tissue using sutures, screws, staples, or any other suitable device that may be installed through holes 155 or loops 156 to prevent lead 150 from moving after installation.

According to one embodiment, operations of each electrode may be programmed by a user (e.g., clinician) via an external controller, such as interrogator 210. For example, a first electrode 160 may be programmed to transmit electrical signals to one or more other electrodes. The sequence, duration, and designation of electrodes as either transmitting electrodes or receiving electrodes may each be programmed by the user. By allowing users to program these operational features of electrodes 160-162, users can manipulate the electric field applied to nerves after implantation.

For example, a user may designate first electrode 160 as the transmitting electrode and second and third electrodes 161, 162 as receiving electrodes. First electrode 160 may be programmed to transmit an electric current pulse to second electrode 161 and third electrode 162 during the same time interval. Alternatively, first electrode 160 may be programmed to transmit a first electric current pulse to second electrode 161 during a first time interval and transmit a second electric current pulse to third electrode 162 during a second time interval. The length of each time interval, sequence of transmission between electrodes, and the current level may each be programmed by a user via an external controller.

According to another embodiment, one or more electrodes may be located within or integral to housing of control module 101 and may be adapted to interact with one or more of the electrodes associated with leads 150. As such, energy may be transmitted between electrode on leads 150 and an electrode on the housing.

By allowing users to program operations of each electrode after implantation of nerve regenerator 100′, the direction, strength, frequency, and oscillating pattern of the electric field may be modified to optimize the therapeutic capabilities of nerve regenerator 100′. Thus, if a particular nerve treatment is not producing desired results, a clinician may simply adjust one or more of the operational parameters associated with the electrodes to modify the electrical stimulation provided to the damaged nerves.

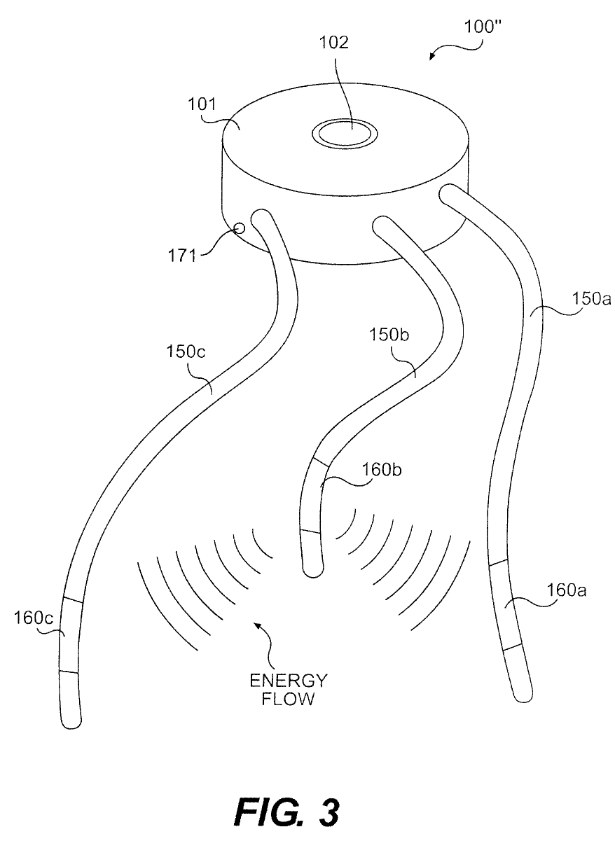

FIG. 3 illustrates an exemplary embodiment of a nerve regenerator 100″ having multiple leads consistent with the disclosed embodiments. As illustrated in FIG. 3, nerve regenerator 100″ may include a plurality of leads 150a-c, each lead including an electrode 160a-c. Leads 150a-c may be implanted within the body of a patient in an area associated with a damaged nerve. After implantation, electrodes 160a-c may be energized to deliver therapeutic electric stimulation to the damaged nerves.

As explained above with respect to FIG. 2, operations of each electrode may be programmed by a user via an external system, such as interrogator 210. Accordingly, users may manipulate the electric stimulation provided by nerve regenerator 100″ to produce a desired oscillating field, such as by modifying the current delivered between a first electrode and any other electrode. Modification to the current delivered can be a change to one or more of amplitude, frequency (if not DC current), period, “off time (e.g. if current flow is not continuous), electrodes receiving energy, or other parameter that would affect the electrical field generated by nerve regenerator 100”.

According to this embodiment, control module 101 may include a power supply communicatively coupled to one or more of electrodes 160a-c. Power may be supplied to electrodes 160a-c sequentially and synchronized by control module 101. As such, power supplied to each of electrodes 160a-c may create an oscillating electromagnetic field between the electrodes. Sequentially energizing electrodes 160a-c may eliminate the need for a separate signal generator for producing the oscillating electromagnetic field to stimulate damaged nerves, thereby reducing cost and power requirements associated with control module 101.

According to one exemplary embodiment, controller 109 of nerve regenerator 100″ may designate a first electrode 160a as a transmitting electrode and one or more other electrodes (e.g., electrodes 160b and 160c) as receiving electrodes. As such, the first electrode 160a may be energized to transmit current to one or more of electrodes 160b and 160c. This current may be provided simultaneously or sequentially, based on a desired pattern for the electric stimulation (e.g., a triangular or other multi-dimensional pattern). It is contemplated that additional electrodes may be included, and that controller 109 may be programmed to selectively energize some or all of the electrodes to create multiple electric field patterns. It is also contemplated that, in certain embodiments that include multiple electrodes, the electrodes may be selectively energized. Accordingly, power may be delivered to energize fewer than the total number of electrodes. As such, current paths, electric field patterns, and other aspects of electric nerve regeneration treatment may be programmed after implantation be designated which electrodes are adapted to transmit and receive electric energy.

Although FIGS. 2 and 3 illustrate embodiments of nerve regenerators that include electrodes 160 disposed along leads 150, it is contemplated that additional and/or different electrode and/or lead configurations may be provided. For example, one or more of nerve regenerator 100′ and 100″ may include an electrode array (such as multi-electrode array 800 of FIG. 8) substituted for or in addition to one or more of leads 150.

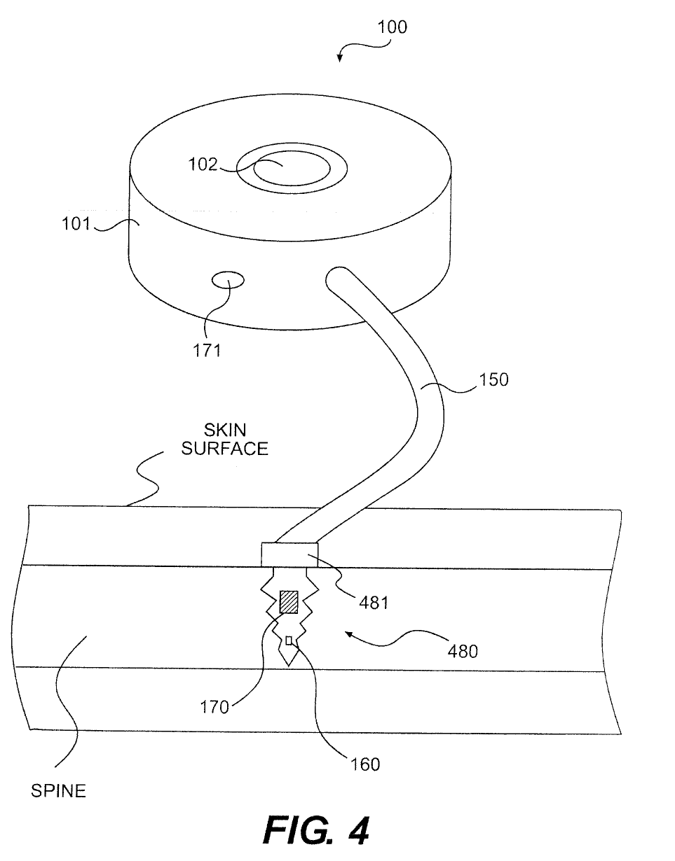

In some embodiments, leads may be customized for implantation within a particular body part, taking certain characteristics of that body part into consideration. For example, if a lead is to be implanted into a bone or other hard tissue (e.g., spine column or skull) of a patient, as illustrated in FIG. 4, a distal end of lead 150 may be customized to include a screw device 480 or other suitable anchoring device, which may be configured to penetrate into the patient's hard tissue or bone. According to one embodiment, screw device 480 may be fixedly engaged with a portion of a patient's spine (e.g., to the pedicle of the spine). Screw device 480 may include self-tapping bone threads or may be inserted into a previously made hole which has been threaded with a standard bone tap. Alternatively screw device 480 may have a sharpened tip for pushing into bone, or may push into a previously made hole in the bone. Screw device 480 may include one or more openings for passing electrode 160 and/or a transducer 170 (e.g., a drug or agent delivery device) into the spinal column of a patient. Screw device 480 may also include a rotating collar 481 that interfaces with lead 150 to allow rotation of lead 150 relative to screw device 480. Accordingly, damage to lead 150 due to twisting or other stresses exerted at the lead-screw interface may be limited.

Alternatively or additionally, screw device 480 may be adapted to include its own transducer 170 and/or electrode 160. Accordingly, wires and/or other conduits (e.g. flow tubes) extending from leads 150 may be hard-wired with integrated electrode 160 and/or transducer 170 of screw 480. In an alternative embodiment, screw device 480 may be adapted to include its own sensor, not shown but preferably a sensor configured to provide information relative to nerve regenerator or other performance measurement of nerve regenerator 100. In yet another embodiment, screw device 480 may include multiple electrodes, functional elements (transducers, sensors, etc.), and/or connection points to connect one or more wires, conduits or other leads to screw device 480.

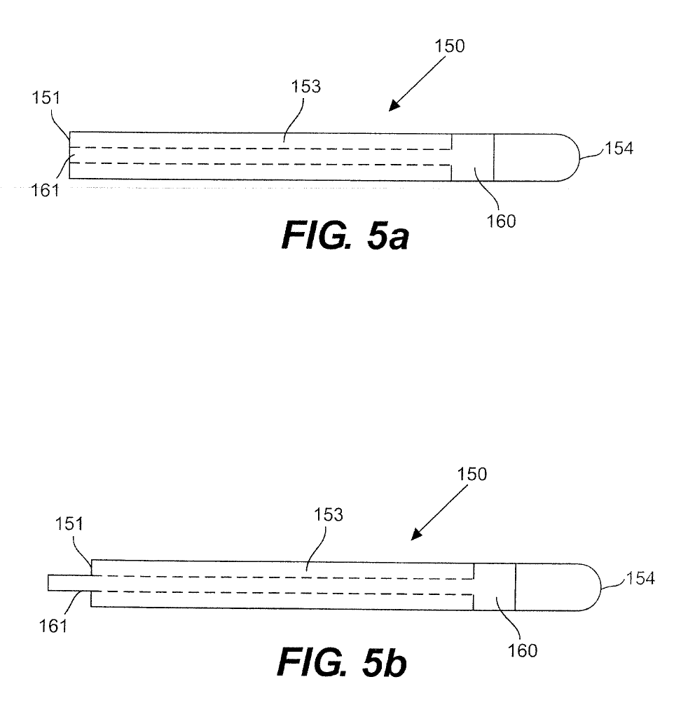

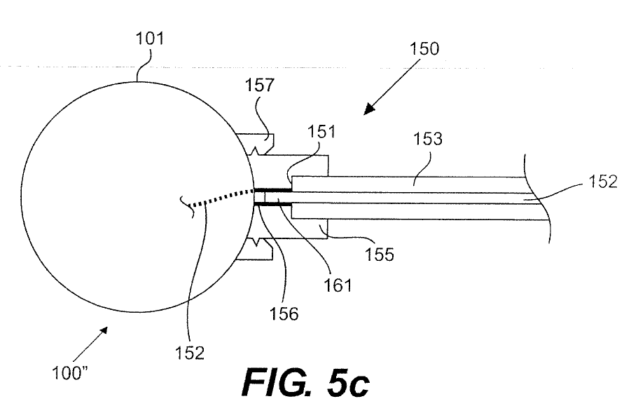

FIGS. 5a-5c illustrate exemplary features associated with lead 150 and its preparation and installation, by a clinician in a sterile field, into control module 101. As shown in FIGS. 5a-5c, lead 150 may include a proximal end 151 and a distal end 154. Proximal end 151 may be adapted for interface with control module 101 of nerve regenerator 100″. Distal end 154 may include one or more holes (not shown) for suturing, screwing, or otherwise anchoring lead 150 to a portion of the patient's body. Distal end 154 may alternatively or additionally include any other anchoring device, such as screw device 480 shown in FIG. 4.

According to one embodiment, lead 150 may be adapted for customized installation during a surgical procedure, thereby allowing surgeons and/or neurologists to customize number, length, and method of placement of lead 150 within a patient. Accordingly, a customized sterile cutting tool may be provided to quickly and precisely cut lead 150, without damaging electrode 160. During placement, the leads may be fully implanted within the body or, alternatively, a distal end of the lead may be implanted, with at least a portion of the lead located external to the body.

By providing leads 150 that may be customized and attached during implantation of nerve regenerator 100″ within the patient's body, leads may be bi-directionally tunnelled under tissue. For example, after placement of the distal end of lead 150, the proximal end may be routed through a surgical tunnel or other guiding device for attachment to control module 101. Prior to the attachment of the proximal end, the lead 150 may be cut to the required length, and terminated with an electrical connector for removable coupled to control module 101. The customizable leads of FIGS. 5a-5c may provide increased flexibility during installation by allowing for bi-directional installation (i.e., installing a either a proximal or distal end first and routing the other end to the desired location). Customizable leads may also limit the amount of coiling of leads left in the body and reduce manufacturing costs related to producing multiple lead lengths with each nerve regenerator 100″.

Lead 150 may be manufactured with built-in electrode 160. Electrode 160 may be integrally-formed with a conductor 161, which may extend to or near proximal end 151 of lead 150 for connection to control module 101 (or one or more of its constituent components). Lead 150 may include an insulation layer 153 (or protective jacket) substantially surrounding conductor 161.

Alternatively or additionally, lead 150 may be manufactured with one or more transducers (not shown) such as, for example, drug delivery mechanism (e.g., needle, plunger, etc.). Accordingly, lead 150 may include an integrally-formed fluid delivery tube (not shown) which may extend to or near the proximal end 151 of lead 150 for connection with control module 101 (or one or more of its constituent components).

As shown in FIG. 5b, leads 150 may be prepared for implantation (e.g. in the sterile field of an operating room or other sterile health care environment) by stripping away a portion of insulation 153 at the proximal end 151 of lead 150, exposing conductor 161 for insertion into header 103 of control module 101.

As illustrated in FIG. 5c, proximal end 151 of lead 150 may be coupled to a snap collet 155 or any other suitable mechanical connector for connecting to control module 101. Snap collet 155 may include an opening for receiving proximal end 151 of lead 150 and a conductive tube 156 for receiving conductor 161 of lead 150. Control module 101 may include a corresponding snap connector 157 configured to mate with a portion of snap collet 155. Conductive tube 156 may be electrically coupled to a wire 156, which may be connected to one or more internal components of control module 101. It is contemplated that leads 150 may be connected to control module using any type of connection device such as, for example, a bayonet lock, compression attachment collar, or any other suitable mechanical or electromechanical connector.

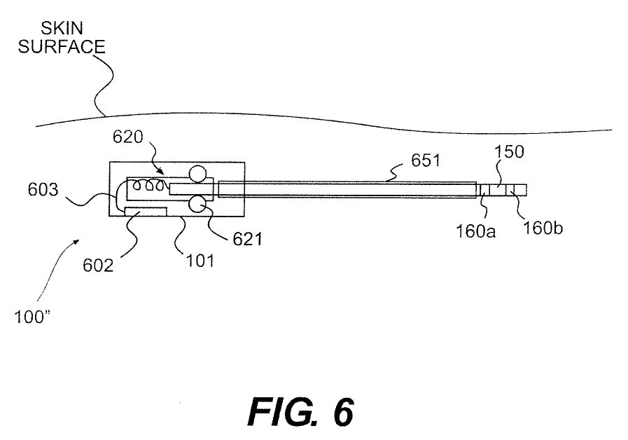

As shown in FIG. 6, nerve regenerator 100″ may include one or more components for extending and/or retracting one or more leads 150 from control module 101. For example, nerve regenerator 100″ may include a linear drive assembly 620 having rollers 621. Rollers 621 may be configured to exert opposing forces against one another with respect to lead 150 so that lead 150 may be securely held by rollers 621. Linear drive assembly 620 may rotate rollers 621, which may, in turn, extend and/or retract lead 150. Although FIG. 6 illustrates drive assembly as a linear drive assembly, other types of drives may be used such as, for example, hydraulic or pneumatic drives activated by accessing a fluid port 102 associated with control module 101. Alternatively, the position of leads 150 may be adjusted by advancing and retracting a wire (e.g., stylet) that can be inserted though the skin and into a portion of lead 150 to manipulate the position of the lead.

Lead 150 may be electrically coupled to power supply 602 via wire bundle 603, which may be coiled so as to provide a sufficient length of wire for extending and/or retracting lead 150. By enabling the extension and retraction of lead 150 after implantation in a patient's body, lead 150 may extend or retract as damaged nerve grows or changes, thereby maintaining an effective positional relationship between electrodes 160a and 160b and the damaged nerve. Extension and retraction of lead 150 may also be performed to improve nerve growth, such as after a sub-optimal growth has been detected by a nerve growth detection assembly of the nerve generator of the present invention. According to one exemplary embodiment, lead 150 may be advanced and retracted as part of a diagnostic process, based on, for example, monitored growth of one or more damaged nerves.

Sheath 651 may be disposed around lead 150. Sheath 651 may be a rigid or semi-rigid material that keeps lead 150 from excessive bending during extension and/or retraction. Sheath 651 may be sutured, screwed, or otherwise secured within the body to hold lead 150 in place after implantation.

According to an exemplary embodiment, lead 150 may include one or more components for controlling the direction of lead 150 to reposition lead 150 (and components associated therewith). For example, lead 150 may include one or more tension elements (e.g., strings, cables, etc.) (not shown) disposed along the length of lead that may be selectively manipulated to hold a portion of lead 150, while other portions of lead 150 are driven by linear drive assembly 620, thereby providing a means for turning, deflecting and/or rotating lead 150.

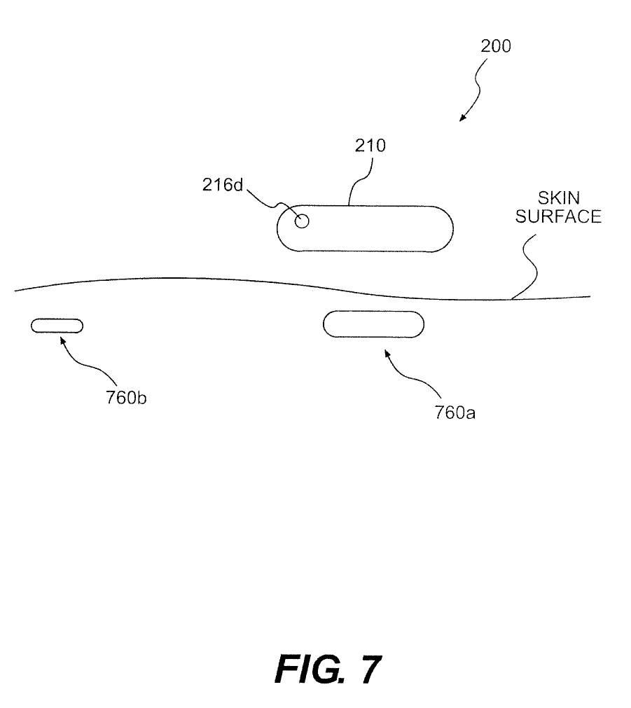

According to one exemplary embodiment, nerve regeneration system 200 may embody a wireless therapeutic delivery system. As illustrated in FIG. 7, nerve regeneration system 200 may comprise one or more wireless electrode components 760a and 760b wirelessly coupled to an external controller, such as interrogator 210. Wireless electrode components 760a and 760b have different construction, and may include self-contained stimulation delivery implants that can be activated by external signals provided by interrogator 210 and/or other control devices (e.g., control module 101 of nerve regeneration 100″). According to one embodiment, wireless electrode 760a includes a power supply and wireless component 760b does not. In another embodiment, wireless electrode 760a includes a wireless receiver/transmitter, and wireless component 760b includes a wireless receiver only. In yet another preferred embodiment, wireless component 760a includes a drug delivery element and wireless component 760b does not. Wireless electrode devices 760a and 760 may also include different sensor or functional elements, different sizes of sensors or functional elements, different sized power supplies, different sized housings, and/or different therapeutic delivery components.

According to one embodiment, wireless electrode components 760a and 760b may each include one or more components for facilitating the administration of therapeutic treatments to damaged nerve tissue. For example, wireless electrode components 760a and 760b may include a microprocessor and associated memory devices for storing treatment parameters provided by interrogator 210 and executing the treatment processes when prompted by interrogator 210.

For example, both of wireless electrode components 760a and 760b may comprise a power supply for generating electric stimulation signals. Additionally, each of wireless electrode components 760a and 760b may include a communication device, such as a wireless transceiver to communicate with interrogator 210 via a wireless communication link (e.g., microwave, RF, infrared, etc.). Accordingly, wireless electrode components 760a and 760b may be configured to receive a command signal from interrogator 210, generate electric stimulation signal in response to the received command, and collect patient data in response to the stimulation. Alternatively or additionally, wireless electrode components 760a and 760b may be configured to receive one or more commands from each other.

One or more wireless electrode components 760a and 760b may be configured to transmit electric current to one or more other wireless electrodes. For example, wireless electrode component 760a may be configured to transmit an electric current to wireless electrode component 760b and/or any additional electrodes (such as electrodes associated with leads 150 of FIGS. 1a, 1b, 2, and 3). Wireless electrode 760b (or other electrodes) may provide an electric current signal to wireless electrodes 760a, thereby creating an electric field such as an oscillating field between the electrodes.

According to one embodiment, wireless electrode component 760a may be further configured as a data collection device for one or more other wireless electrode component. As such, wireless electrode component 760a may be adapted to receive/collect patient data from one or more other wireless electrode components and provide the patient data to interrogator 210. As such, wireless electrode component 760a may include one or more memory devices for storing patient data.

In addition to providing electric stimulation, one or more of wireless electrode components 760a and 760b may be configured to deliver other types of nerve regeneration treatments. For example, at least one of wireless electrode components 760a and 760b may include an on-board fluid delivery device (e.g., a pump, a reservoir, etc.) for delivering therapeutic fluid as part of a nerve regeneration treatment.

FIG. 8 illustrates an exemplary multi-electrode array 800 that may be implemented with one or more of the disclosed embodiments. Multi-electrode array 800 may include a substrate made of, for example, durable biocompatible material (e.g., silicon), and a plurality of sharpened projections 820 that may project from the substrate and contact with or extend into an area of the body associated with one or more damaged nerves. Substrate may include electronics, e.g. power supply or power receiving means, signal processing circuitry such as analog to digital conversion and/or signal multiplexing, and other electronic circuitry.

Each projection 820 may have an active electrode 810 at its distal tip and may be electrically isolated from neighboring projection 820 by a suitable non-conducting material. In an exemplary embodiment, one or more projections 820 may include multiple electrodes 810 along its length. In another exemplary embodiment each projection is approximately 0.5-5.0 mm long. In yet another exemplary embodiment, each projection is configured to be inserted into the cortex of the brain, into the spinal cord and/or into a peripheral nerve of a patient. Also, the array 800 may include different types of electrodes or other functional elements, such as, for example, recording electrodes, stimulating electrodes, photo or other sensors, acoustic or other transducers, or any combination thereof. Alternatively or additionally, the differences between electrode types may include different materials of construction, coatings, thicknesses, geometric shapes, etc. Each of the electrodes 810 may form a recording channel that may directly detect electrical signals generated from single cells such as a neuron in the electrode's vicinity. Further signal processing may isolate the individual neuron signals. Alternatively or additionally, while the electrodes 810 may detect multiple individual cellular signals, only a particular subset of the electrodes 810 may be selectively chosen for further processing. A suitable preprocessing method, such as, for example, a calibration or configuration process, may be used to selectively choose the subset of the electrodes 810.

According to one embodiment, microelectrode array 800 may include a plurality of longitudinal projections 820 extending from a base. The projections may be rigid, semi-flexible or flexible, the flexibility such that each projection can still penetrate into neural tissue, potentially with an assisting device or with projections that only temporarily exist in a rigid condition. The microelectrode array may be inserted into the brain, preferably using a rapid insertion tool, such that the projections pierce into the brain and the base remains in close proximity to or in light contact with the surface of the brain. At the end of each projection is an electrode. In alternative embodiments, electrodes can be located at a location other than the tip of the projections or multiple electrodes may be included along the length of one or more of the projections. One or more projections may be void of any electrode, such projections potentially including anchoring means such as bulbous tips or barbs, not shown.

The electrodes may configured to detect electrical brain signals or impulses, such as individual neuron spikes or signals that represent clusters of neurons such as local field potential (LFP) and electroencephalogram (EEG) signals. Each electrode may be used to individually detect the firing of multiple neurons, separated by neuron spike discrimination techniques. Other applicable signals include electrocorticogram (ECoG) signals and other signals, such as signals between single neuron spikes and EEG signals. The microelectrode array may be placed in any location of a patient's brain allowing for the electrodes to detect these brain signals or impulses. In a preferred embodiment, the electrodes can be inserted into a part of the brain such as the cerebral cortex (e.g. an electrode array with projections approximately 1.0-1.5 mm long, with electrodes at the tip of each projection). Alternative forms of penetrating electrodes, such as wire or wire bundle electrodes, can make up or be a component of the sensor of the present invention. The various forms of penetrating electrodes described above can be placed into tissue within or outside of the patient's cranium, such tissue including but not limited to: nerve tissue such as peripheral nerve tissue or nerves of the spine; organ tissue such as heart, pancreas, liver or kidney tissue; tumor tissue such as brain tumor or breast tumor tissue; other tissue and combinations of the preceding, The electrodes are preferably configured to both record signals as well as transmit signals and/or energy.

The microelectrode array may include one or more projections with and without electrodes, both the projections and electrodes having a variety of sizes, lengths, shapes, surface areas, forms, and arrangements. The microelectrode array may be a linear array (e.g., a row of electrodes) or a two-dimensional array (e.g., a matrix of rows and columns of electrodes such as a ten by ten array), or wire or wire bundle electrodes, all well known to those of skill in the art. An individual wire lead may include a plurality of electrodes along its length. Projections and electrodes may have the same materials of construction and geometry, or there may be varied materials and/or geometries used in one or more electrodes. According to one embodiment, electrodes may measure approximately 200 micrometers in diameter at the base, approximately 40-50 micrometers in diameter at the midpoint, and approximately 12-14 micrometers at the tip. It is contemplated that additional and/or different diameter electrodes may be used. Each projection and electrode is configured to extend into tissue to detect one or more cellular signals such as those generated form the neurons located in proximity to each electrode placement within the tissue.

In addition to monitoring data, electrode array 800 and/or one or more electrodes 810 associated therewith may be adapted to deliver electromagnetic energy for stimulating one or more damaged nerves or nerve tissue. Furthermore, it is contemplated that one or more electrodes 810 may be designated to provide therapeutic stimulation, while one or more other electrodes may be designated as sensor electrodes dedicated to monitoring one or more biological, physiological, chemical, and/or electrical characteristics associated with the patient's body.

Electrode array 800 may include a wire bundle 830 that provides one or more conductors for coupling electrodes to a controller, such as control module 101 shown in FIGS. 1a and 1b. Wire bundle 830 may include, for example, one conductor per electrode. Alternatively, wire bundle 830 may include a limited number of conductors, each conductor electrically connected to multiple electrodes and configured to deliver energy or communicate data with a plurality of electrodes. Accordingly, each conductor may be coupled to a hardware or software controller associated with control module 101 for routing signals to the appropriate electrode.

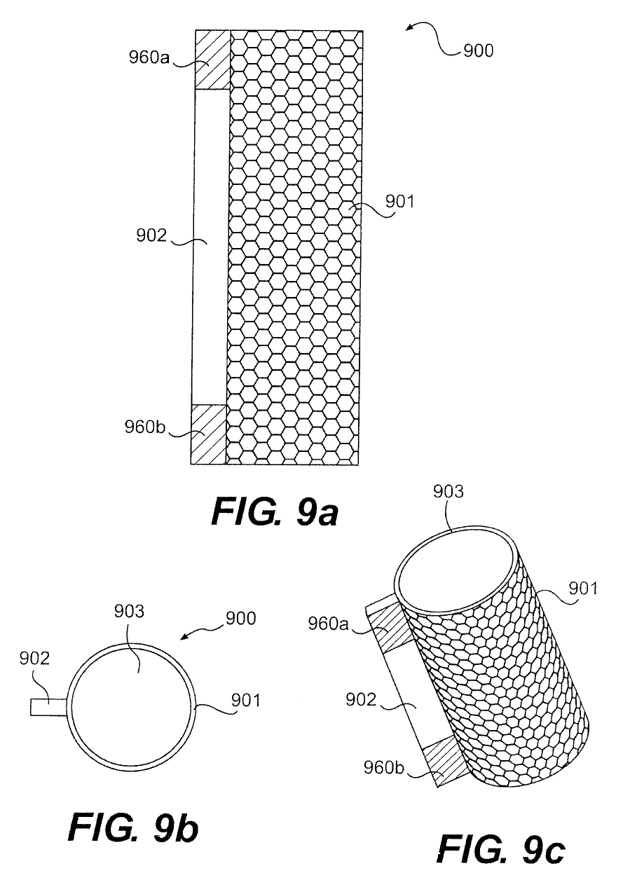

FIGS. 9a-9c provide side, end, and perspective views, respectively, of an exemplary structure 900 for enhancing and controlling the direction of nerve growth consistent with the disclosed embodiments. According to one embodiment, structure 900 may be a standalone implantable therapeutic device associated with nerve regeneration system 200 of FIG. 7, which, like nerve regenerator 100″, may be wirelessly coupled to interrogator 210. Structure 900 may be particularly advantageous to repair severed spinal nerves where the direction of nerve re-growth and/or nerve re-connection must be precisely controlled (e.g., to repair a severed nerve or reconnect a nerve to another nerve or a particular muscle or gland).

Structure 900 may comprise a tubular member 901 that may be placed around a portion of a diseased, damaged or severed nerve and may provide a channel for promoting growth of the nerve within structure 900. In addition to supporting and guiding the growth of the damaged nerve, structure 900 may include one or more components for delivering therapeutic stimulation within tubular member 901. For example, structure 900 may include a plurality of electrodes 960a, 960b for providing electric stimulation to the damaged nerve and a controller 902 for controlling the operation of electrodes 960a and 960b. Although FIG. 9a illustrates structure 900 as containing two electrodes, additional electrodes may be provided depending upon the length of structure 900. For example, one or more additional electrodes may be provided between electrodes 960a and 960b. Alternatively or additionally, additional electrodes may be located in multiple positions around structure 900 (e.g., two electrodes provided on opposing sides for 180-degree separation, four electrodes with 90-degree separation, or multiple electrodes with asymmetric positioning. The nerve growth scaffold of tubular member 901, combined with the electric field generated by passing current between electrodes 960a and 960b (e.g. from a DC constant current of approximately 200-1000 microamps that turns off and/or switches direction after a period of time greater than 30 seconds) enhances nerve growth and the resultant patient recovery.

Alternatively or additionally, one or more wireless electrode components (such as electrode components 760a or 760b of FIG. 7) may be employed in conjunction with or as an alternative to electrodes 960a and 960b. Because electrodes 760a and 760b may be adapted for percutaneous delivery, the electric field treatment capabilities of structure 900 may be modularly expended based on the effectiveness of nerve regeneration treatments.

Tubular member 901 may embody a hollow, flexible mesh cylinder. As illustrated in FIG. 9b, tubular member 901 forms a nerve growth channel 903 that provides an area for concentrating and guiding the growth of the damaged nerve. Tubular member 901 may be constructed of a polymeric foam material arranged in a lattice-type structure. According to one embodiment, tubular member 901 may be constructed of bioabsorbable material, which may break down and dissolve within the body in a predetermined amount of time. Because tubular member 901 may naturally dissolve in the body after use, the need for additional invasive surgery to remove tubular member 901 may be eliminated.

A portion of tubular member 901 may extend at least partially into nerve growth channel 903 to provide a structural element within nerve growth channel to provide a guide for supporting and promoting nerve growth within nerve growth channel 903.

Tubular member 901 may be coated or soaked in a chemical (drug or other agent) and/or combined with stem cells for enhancing or stimulating the growth of the damaged nerve. For example, tubular member 901 may be coated with a chemical that is configured to release over time as the tubular member 901 dissolves. Alternatively or additionally, different chemicals may be deposited in different layers, so that different chemicals can be released at different times.

Controller 902 may be electrically coupled to electrodes 960a and 960b. Controller 902 may include a power source (e.g., battery, etc.) (not shown) for supplying power to electrodes 960a and 960b to generate electric stimulation signals. Controller 902 may also include a wireless transceiver (not shown) for receiving command signals from and communicating data with interrogator 210. As such, users may adjust the timing, sequence, and duration of alternating electric pulses between electrodes 960a and 960b. As the nerve grows, users may modify the timing, sequence, and duration of the pulses based on the effectiveness of the nerve treatment.

Controller 902 may also include one or more fluid delivery devices (not shown) for delivering therapeutic fluids to nerve growth channel 903. For example, controller 902 may include a reservoir, a pump, and one or more needles or other fluid delivery elements that protrude from controller 902 through a wall of tubular member 901. Accordingly, controller 902 may administer therapeutic fluid (e.g., nerve growth factor) to a damaged nerve growing within nerve growth channel 903.

According to one embodiment, controller 109 may be coupled to one or more chambers (not shown) that may include an electrically-charged substance (e.g., therapeutic or diagnostic fluid, stem cells, etc.). When small-signal electric signals are applied to the one or more chambers a repelling force may cause the electrically charged substances to be released into nerve growth channel 903 via a process known as iontophoresis.

Structure 900 may be configured to operate in either manual mode or automated mode. In manual mode, operation of structure 900 and/or controller 109 is controlled by a user via interrogator 210. In automated mode, controller 109 may include one or more software or hardware programmable routines that monitor neural responses to nerve regeneration treatments and automatically adjust nerve treatment parameters, based on the monitored responses. For example, controller 109 may be configured to automatically adjust a drug delivery or electric treatment parameters if monitored nerve growth deviates from a predetermined nerve growth level.

According to one embodiment, structure 900 may be implanted between opposite ends of a severed nerve to promote direct reconnection of the ends of the nerve. A user (via interrogator 210) may initiate therapeutic electric treatments and monitor the growth of the nerves (e.g. via the Internet) based on the treatments. As the nerve treatment progresses, a user may monitor the growth of the nerve and modify the timing, sequence, and duration of the pulses to maximize the effectiveness of the treatment on the nerve growth.

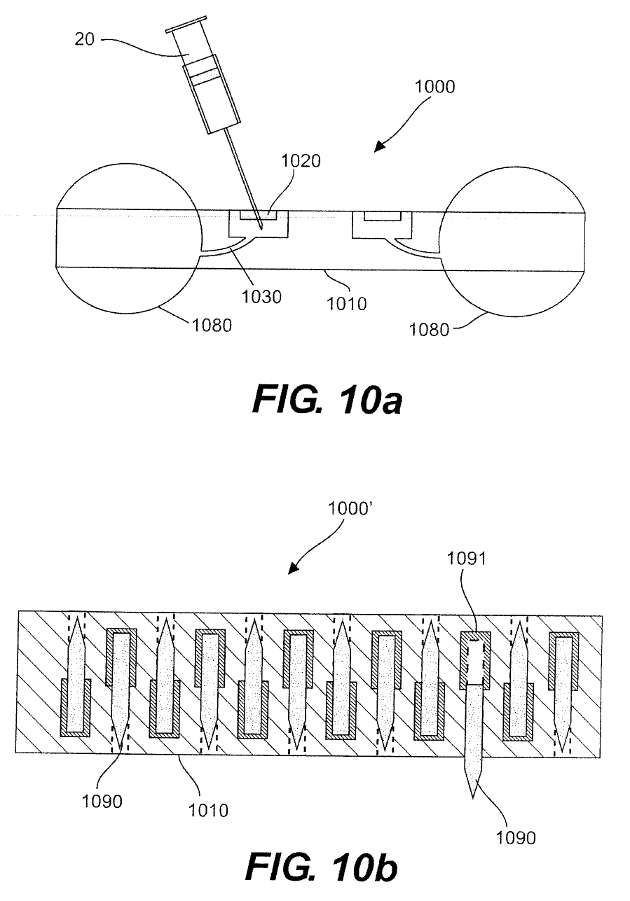

In some cases, it may be advantageous to apply physical stimulation of damaged nerve tissue to enhance the effectiveness of nerve regeneration treatments. FIGS. 10a and 10b illustrate exemplary tissue manipulating devices 1000 and 1000′ that may be implanted within the body of the patient. Tissue manipulating devices 1000 and 1000″ may be configured to physically manipulate, traumatize, disrupt, and/or otherwise stimulate tissue around nerve regenerator 100″ for aiding in the efficacy of other nerve regeneration stimulation and/or to provide stand-alone treatment for promoting nerve regeneration. Moreover, tissue manipulating devices 1000 and 1000″ may be configured to mimic the proliferative response often encountered with surgical procedures. The manipulation and forces applied by devices 1000 and 1000′ to the damaged nerves and the neighboring tissue, provides the stimulus to cause and/or enhance nerve regeneration.

According to one embodiment, tissue manipulating devices 1000 and 1000′ may be provided as an attachment or accessory to nerve regenerator 100″ or as an integrated component of nerve regenerator 100″. Alternatively, tissue manipulating devices 1000 and 1000′ may be configured as standalone implantable devices.

As shown in FIG. 10a, tissue manipulating device 1000 may include a sealed housing 1010 that includes a port 1020 for receiving fluid. Port 1020 may be in fluid communication with an expandable member (e.g., balloons 1080) via a tube 1030, each of which may be at least partially disposed within housing 1010. Expandable members, such as balloons 1080, may be compliant and/or non-compliant balloons, and may embody angioplasty balloon construction and/or other surgical-grade expandable elements.

As illustrated in FIG. 10a, a syringe 20 may be used to inject a suitable fluid (e.g., air, saline, water, etc.) into port 1020 to inflate balloons 1080. Similarly, syringe 20 may be used to withdrawal fluid from port 1020 to deflate balloons 1080. Inflating and deflating balloons 1080 may stretch, compress, contract, tear, split, massage, and/or otherwise apply forces configured to stimulate nerve tissue. Alternatively or additionally, injection of fluid into port 1020 may cause an articulating member (not shown), to move and similarly apply forces to neighboring tissue such as to achieve or enhance nerve regeneration. In some cases, tissue receiving these applied forces may respond more effectively to nerve regeneration treatment (e.g., drug treatment, electric stimulation treatment, etc.). It is also contemplated that expandable members (e.g., balloons 1080) may include projecting elements (e.g., needles, scalpels, cages, other balloons, etc.) disposed on the surface of expandable member to provide additional manipulation or disruption of and/or interaction with the surrounding tissue. It is also contemplated that balloons 1080 may be irregularly shaped. It is also contemplated that expandable member may include one or more electrodes or other devices for delivering nerve regeneration treatment to damaged nerve tissue.

According to one embodiment, housing 1010 and/or syringe 20 may include a pressure or volumetric indicator to display an amount of fluid within expandable member. This information may provide a user with an indication of the amount of stimulation and/or force being applied to the surrounding tissue.

Although tissue manipulating device 1000 is illustrated in FIG. 10a as being manually operated, tissue manipulating device 1000 may also be configured for automated use. For example, sealed housing 1010 may include a controller (not shown) coupled to a fluid delivery system (not shown) that includes a reservoir for storing fluid for inflating and/or balloons 1080 and a pump (not shown) for controlling fluid flow to the balloons 1080. The controller may include a transceiver and may be configured to activate tissue manipulating device 1000 in response to command signals received from interrogator 210 and/or control module 101 associated with nerve regeneration system 200.

As illustrated in the alternate embodiment shown in FIG. 10b, tissue manipulating device 1000′ may include a housing 1010 having a plurality of projecting elements 1090 coupled to a drive assembly 1091. Elements 1090 may be extended and retracted from the housing via the drive assembly 1091.

Drive assembly 1091 may include, for example, a hydraulic or pneumatic drive, a micro-stepper motor, a MEMs driver, screw-type actuator, magnetic driver, or any other suitable device for driving projecting elements 1090 into the surrounding tissue.

Projecting elements 1090 may include symmetric or asymmetric sharpened and/or blunt tips that, when extending from housing 1010, may apply forces to the nerve tissue adjacent to housing 1010. According to one aspect, projecting elements 1090 may include a sensor (e.g., an optical sensor for measuring depth of projecting elements, a temperature sensor, a heart-rate monitor, a single cell electrical sensor, an EKG, EMG, ECoG, LFP or EEG sensor, etc.) for collecting patient data. Alternatively, projecting elements 1090 may include a nerve stimulation device (e.g., a drug or other agent delivery device or an electrode) for delivering nerve stimulation while projecting elements 1090 are extended.

One or more of tissue manipulating devices 1000 or 1000′ may be coupled to a portion of nerve regenerator 100″. For example, housing 1010 of tissue manipulating device 1000′ may be coupled to a housing of control module 101. Drive assembly 1091 may be electrically coupled to controller 109 of control module 101 of FIG. 1b. During nerve regeneration treatments, controller 109 may provide command signals to drive assembly 1091, which may, in turn, actuate projecting elements 1090 to provide physical stimulation of the tissue adjacent to control module 101. In an exemplary embodiment, drive assembly 1091 includes a magnetic drive assembly including multiple electromagnets configured to advance and retract projecting elements 1090 in precise increments. Alternatively or additionally, drive assembly 1091 may include a pneumatic or hydraulic piston which is operably attached to projecting element 1090 for controllable advancement and retraction of projecting element 1090. Alternatively or additionally, drive assembly 1091 may include a lead screw drive which is operably attached to projecting element 1090 for controllable advancement and retraction of projecting element 1090.

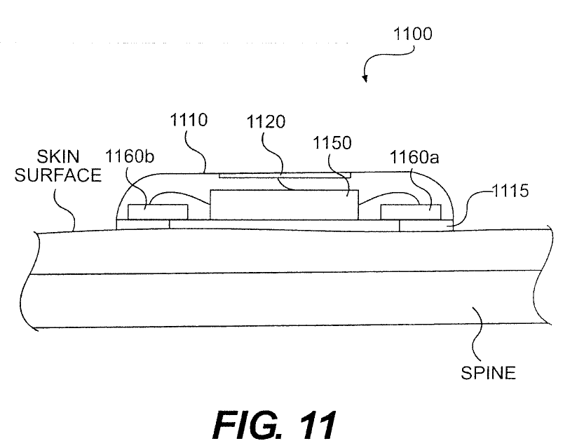

According to one embodiment, nerve regeneration system 200 may also be configured to provide magnetic stimulation to damaged nerve tissue. FIG. 11 illustrates an exemplary magnetic therapeutic device 1100 that may be employed as part of nerve regeneration system 200 to deliver magnetic stimulation to damaged nerve tissue to enhance nerve regeneration treatments.

Magnetic therapeutic device 1100 may include a housing 1110, a signal generator 1120, a battery 1150, and one or more electromagnets 1160a, 1160b for producing a therapeutic magnetic field. Magnetic therapeutic device 1100 may also include an adhesive device 1115 (e.g., adhesive pads such as an adhesive pad integral to an EKG lead, bandages, etc.) for temporarily securing device 1100 to a portion of a patient's body. For example, as illustrated in FIG. 11, magnetic therapeutic device 1100 may be attached to the back of a patient undergoing nerve regeneration treatment for a spinal cord injury. Magnetic therapeutic device 1100 may include additional, fewer, and/or different components than those listed above. For example, magnetic therapeutic device 1100 may include communication electronics for communicating nerve treatment data and/or patient data with external diagnostic tool, such as interrogator 210.

Battery 1150 may be disposed within housing 1110 and configured to provide a power output for operating one or more devices associated with magnetic therapeutic device 1100. For example, battery 1150 may be configured to provide power for operating signal generator 1120 that, in turn, energizes electromagnets 1160a and 1160b to produce a therapeutic magnetic field. In an exemplary embodiment, electromagnets 1160a and 1160b are energized in a first polarity for a first time period, and a second polarity for a second time period. The first and second time periods are preferably at least 30 seconds.

Signal generator 1120 may be an electronic assembly configured to manipulate the desired magnetic field associated with each of electromagnets 1160a and 1160b. For example, signal generator 1120 may include switching and control circuitry that manipulates the DC power provided by battery 1150 to produce a variable electric field for energizing electromagnets 1160a and 1160b. According to one embodiment, signal generator 1120 may switch battery 1150 between on and off states to produce the variable electric field required to produce a magnetic field.

Electromagnets 1160a and 1160b may be configured to receive pulsed electric energy from battery 1150 and generate a concentrated magnetic field proportional to the electrical energy. According to one embodiment, electromagnets 1160a and 1160b may embody a conductor wound around an iron core. Electric energy may be provided by signal generator 1120 to the conductor. The energy may be stored and/or directed, using the iron core, to produce a magnetic field on the face of the iron core. Electromagnets may be energized to produce the same polarity, opposing polarity, or may be alternately energized to sequentially produce varying magnetic fields.

According to one exemplary embodiment, one or more magnets (e.g., electromagnets 1160a or 1160b or, alternatively, additional magnetic devices) may be attached to a rotatable substrate (not shown) within housing 1110. The rotatable substrate may be coupled to signal generator 1120 and may be configured to rotate in order to vary the magnetic field provided by the magnets. This rotation rate, speed, and/or frequency may be controller by signal generator 1120. The rotatable substrate may be rotated by a stepper motor assembly, magnetic drive assembly, hydraulic or pneumatic drive assembly, or any other mechanism suitable for rotating the substrate.

Magnetic therapeutic device 1100 may provide magnetic therapy to regenerate or enhance regeneration of damaged nerves in or near the spinal cord of a patient. According to one embodiment, magnetic therapeutic device 1100 may be operated remotely by a clinician using interrogator 210 to selectively provide magnetic stimulation during nerve regeneration treatment. Alternatively, magnetic therapeutic device 1100 may be automatically controlled by interrogator 210 as part of a closed loop diagnostic system. Accordingly, magnetic therapeutic device 1100 may be automatically operated if, for example, nerve growth is enhanced by the application of magnetic therapy.

According to one embodiment, magnetic therapeutic device 1100 may include one or more electrodes (not shown) or may be adapted for coupling to one or more leads 150 associated with nerve regenerator 100″ of FIG. 1. As such, magnetic therapeutic device 1100 may be integrated as part of nerve regeneration system 200.

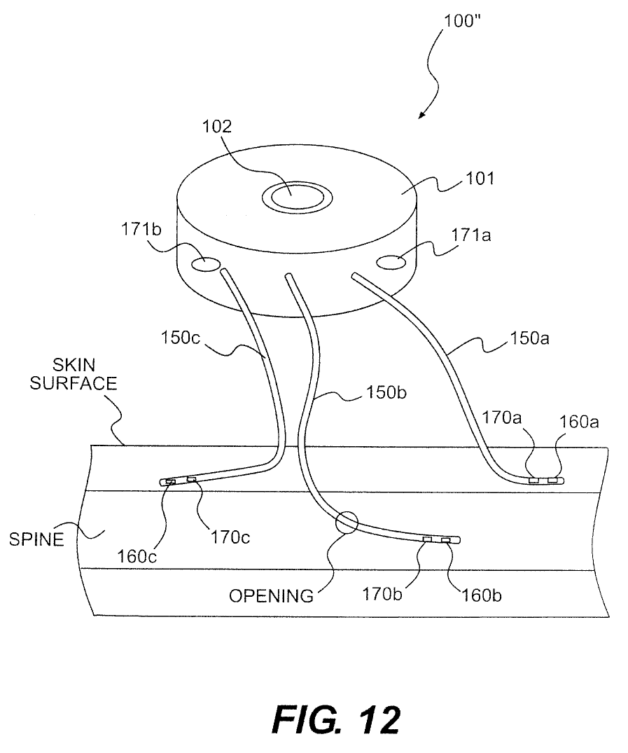

FIG. 12 illustrates an exemplary configuration of nerve regenerator 100″ consistent with the disclosed embodiments. For example, nerve regenerator 100″ may be configured with multiple leads 150a-c, each lead 150a-c being strategically placed percutaneously into the body so as to provide nerve regeneration therapy to multiple areas of the body. FIG. 12 also illustrates an exemplary method of treating a patient with a spinal cord injury. One or more leads 150a-c may be disposed proximate to but outside the spine to deliver therapeutic treatments to damaged nerves proximate the implantation site (primarily the posterior side of the spine). In addition, one or more additional leads 150a-c may be inserted in the spine of the patient to provide nerve regeneration therapy to damaged nerves proximate that implantation site. Alternatively or additionally, additional leads (not shown), may be placed at a location on the anterior side of the spine. Lead placement may be chosen to maximize regeneration of afferent nerves (sensors or receptor neurons), and/or efferent nerves (motor or effector neurons).

Each of leads 150a-c may include a respective electrode 160a-c and transducer 170a-c (e.g., a drug or other agent delivery device) that may deliver electric stimulation treatment coupled with therapeutic drug treatments. Further, as explained above, electrodes 160a-c may be sequentially and/or synchronously energized to provide a desired therapeutic oscillating electric field. Also as explained, each of electrodes 160a-c may embody sensors or other data monitoring devices that are configured to collect patient data associated with a biological, physiological, chemical, and/or electrical response to the nerve regeneration therapies. The monitored data may be used by regenerator 100″ and/or interrogator 210 (in a closed-loop system) and/or a physician, health technician, and/or trained patient (in a “manual” operating mode) to modify and/or customize treatments in response to the monitored patient data. In an exemplary embodiment, one or more of transducer 170a-c is a drug or other agent delivery device including an output port fluidly connected to the distal end of a conduit, such as a capillary tube. The conduit is fluidly attached on its proximal end to a pressurized reservoir and/or pumping assembly. The reservoir or pumping assembly is refillable via injection port 102.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

In addition, where this application has listed the steps of a method or procedure in a specific order, it may be possible, or even expedient in certain circumstances, to change the order in which some steps are performed, and it is intended that the particular steps of the method or procedure claim set forth here below not be construed as being order-specific unless such order specificity is expressly stated in the claim.

1. A method for treating a body comprising:
implanting an elongated lead within a patient's body, the elongated lead having a plurality of electrodes configured to deliver electric stimulation to an area of the patient's body;
selecting at least one transmitting electrode from among the plurality of electrodes; and
causing the at least one transmitting electrode to transmit an electric signal to one or more other electrodes to stimulate a damaged nerve.

2. The method of claim 1, further comprising implanting the elongated lead proximate the patient's spine.

3. The method of claim 1, wherein selecting the at least one transmitting electrode includes:
determining a location of the damaged nerve; and
selecting the at least one transmitting electrode based on the determined location.

4. The method of claim 1, further comprising monitoring the patient's response to the electric signal.

5. The method of claim 1, further comprising causing the at least one electrode to transmit the electric signal to a first receiving electrode during a first time interval and transmit the electric signal to a second receiving electrode during a second time interval.

6. The method of claim 1, further comprising causing the at least one electrode to simultaneously transmit the electric signal to a first receiving electrode and a second receiving electrode.

7. The method of claim 1, further comprising modifying at least one parameter associated with the plurality of electrodes to modify the electric stimulation applied to the area of the patient's body.

8. (canceled)

9. The method of claim 1, further comprising securing at least a portion of the lead proximate the area of the patient's body.

10. The method of claim 1, wherein causing the at least one transmitting electrode to transmit electric energy includes providing a command signal to a controller, wherein the controller is configured to energize the at least one transmitting electrode in response to the command signal.

11. (canceled)

12. The method of claim 1, wherein the electric signal comprises an electric current pulse.

13. A system used for a nerve regeneration treatment, comprising:
a controller;
an elongated lead configured to be implanted within a patient's body; and
a plurality of electrodes disposed along the elongated lead and configured to deliver electric stimulation to an area of a patient's body, the plurality of electrodes comprising at least one transmitting electrode in communication with the controller,
wherein the at least one transmitting electrode is configured to transmit an electric signal to one or more other electrodes, and
wherein the controller is configured to control operation of the at least one transmitting electrode.

14. The system of claim 13, wherein the electric signal comprises an electric current pulse.

15. The system of claim 13, wherein the controller controls at least one of: direction, strength, frequency, and oscillating pattern of an electric field applied to the area.

16. The system of claim 13, wherein the controller controls at least one of: duration of the transmission; sequence of the transmission between electrodes; and signal level of the transmission.

17.-18. (canceled)

19. The system of claim 13, wherein the controller is configured to designate the transmitting electrode from the plurality of electrodes.

20. (canceled)

21. The system of claim 13, wherein the at least one transmitting electrode is configured to simultaneously transmit the electric signal to a first receiving electrode and a second receiving electrode.

22. The system of claim 13, wherein the at least one transmitting electrode is configured to transmit the electric signal to a first receiving electrode during a first time interval and transmit the electric signal to a second receiving electrode during a second time interval.

23. The system of claim 13, wherein at least one of the plurality of electrodes is configured to monitor the patient's response to the applied nerve generation treatment.

24. The system of claim 23, wherein the controller is configured to modify at least one parameter associated with the plurality of electrodes to modify the electric stimulation applied to the area of the patient's body.

25. (canceled)

26. The system of claim 13, wherein the lead comprises a securing device configured to secure at least a portion of the lead proximate the area of the patient's body.

27. A method for treating a body comprising:
implanting a first elongated lead in a patient's body, the first elongated lead having a first electrode;
implanting a second elongated lead within a patient's body, the second elongated lead having a second electrode; and
sequentially energizing the first and second electrodes to create an oscillating electromagnetic field between the electrodes.

28. The method of claim 27, wherein the first electrode is configured to transmit an electric signal to the second electrode.

29. The method of claim 28, further comprising selecting the first electrode from among a plurality of electrodes.

30. The method of claim 27, further comprising monitoring the patient's response to the electromagnetic field.

31. The method of claim 27, further comprising modifying at least one parameter associated with the first and second electrodes to modify the oscillating electromagnetic field.

32. The method of claim 27, further comprising detecting, by at least one of the first and second electrodes, electrical signals from the patient's body.

33. The method of claim 27, further comprising adjusting the energization of the first and second electrodes by adjusting at least one of: direction, strength, frequency, and oscillating pattern of the electromagnetic field.

34. The method of claim 27, further comprising adjusting the energization of the first and second electrodes by adjusting at least one of: duration of energization; sequence of energization of the electrodes; and power level of the energization.

35. The method of claim 27, further comprising securing at least a portion of the first and second leads within the patient's body.

36. The method of claim 27, wherein the first and second electrodes are coupled to a controller for energizing the first and second electrodes.

37. The method of claim 36, wherein sequentially energizing first and second electrodes comprises:
energizing, by the controller, the first electrode; and
energizing, by the first electrode, the second electrode.

38. A system used for a nerve regeneration treatment, comprising:
a first elongated lead configured to be implanted within a patient's body and having a first electrode;
a second elongated lead configured to be implanted within the patient's body and having a second electrode; and
a controller configured to sequentially energize the first and second electrodes to create an oscillating electromagnetic field between the electrodes.

39. The system of claim 38, wherein the controller is configured to energize only the first electrode, and the first electrode is configured to transmit electric energy to the second electrode.

40. The system of claim 39, wherein the controller is configured to designate the first electrode from a plurality of electrodes.

41. The system of claim 38, wherein the controller controls at least one of: direction, strength, frequency, and oscillating pattern of the electromagnetic field.

42. The system of claim 38, wherein the controller controls at least one of: duration of the energization; sequence of the energization between electrodes; and power level of the energization.

43.-44. (canceled)

45. The system of claim 38, wherein at least one of the plurality of electrodes is configured to monitor the patient's response to the oscillating electromagnetic field.

46. The system of claim 38, wherein the controller is configured to modify at least one parameter associated with the first and second electrodes to modify the oscillating electromagnetic field applied to the patient's body.

47. The system of claim 38, wherein at least one of the first and second electrodes is configured to detect electrical signals from the patient's body.

48. (canceled)

49. A method for treating a body comprising:
implanting at least a portion of an elongated lead in a patient's body, a distal end of the elongated lead comprising an anchoring device;
securing, by the anchoring device, the distal end of the elongated lead to a portion of a patient's body.

50.-51. (canceled)

52. The method of claim 50, wherein the connecting member comprises a snap fastener.

53. The method of claim 49, wherein the elongated lead comprises at least one of an electrode, a transducer, and a sensor located proximate to the anchoring device.

54. The method of claim 53, wherein the anchoring device comprises the electrode and the transducer.

55. The method of claim 54, wherein the at least one of the electrode and the transducer is integrally formed with the anchoring device.

56.-57. (canceled)

58. A nerve generation system, comprising:
a controller housing;
an elongated lead extending from the housing, at least a portion of the elongated lead being configured to be implanted within a patient's body; and
an anchoring device located at a distal end of the elongated lead, the anchoring device being configured to secure the distal end of the elongated lead to a portion of the patient's body.

59. The system of claim 58, further comprising at least one of an electrode, a transducer, and a sensor located proximate the anchoring device.

60. (canceled)

61. The system of claim 59, wherein the at least one of the electrode and the transducer is integrally formed with the anchoring device.

62.-64. (canceled)

65. The system of claim 64, wherein the connecting member comprises a snap fastener.

66. A method for treating a body comprising:
implanting at least a portion of an elongated lead in a patient's body, the elongated lead having at least one of an electrode and a transducer disposed thereon, wherein the elongated lead is moveably coupled to a controller housing that includes a driver assembly; and
causing a driver assembly to move the elongated lead relative to the controller housing.

67. (canceled)

68. The method of claim 66, further comprising determining a location of a damaged nerve.

69. The method of claim 68, further comprising causing the driver assembly to extend the elongated lead proximate the damaged nerve.

70. The method of claim 69, further comprising delivering a nerve regeneration treatment to the damaged nerve.

71. The method of claim 70, wherein the elongated lead includes at least one electrode, the method further comprising energizing the electrode to deliver a therapeutic electric signal to the damaged nerve.

72. The method of claim 70, wherein the elongated lead includes a plurality of electrodes, the method further comprising sequentially energizing the plurality of electrodes to create an oscillating electromagnetic field between the electrodes.

73. A nerve generation system, comprising:
a controller housing;
an elongated lead movably coupled to the housing, at least a portion of the elongated lead being configured to be implanted within a patient's body; and
at least one of an electrode and a transducer coupled to the elongated lead,
wherein the controller housing comprises a driver assembly configured to move the elongated lead relative to the controller housing.

74.-77. (canceled)

78. A method for treating a body comprising:
implanting a first wireless electrode device in a patient's body proximate a damaged nerve;
implanting a second wireless electrode device that is different in configuration from the first wireless electrode device;
causing at least one of the first and second wireless electrode devices to administer a nerve regeneration treatment to the damaged nerve; and
providing data indicative of the patient response to an external controller.

79. The method of claim 78, further including storing the patient data in a memory module associated with at least one of the first and second wireless electrode devices.

80. The method of claim 78, further comprising implanting a second wireless device within the patient's body, the second wireless electrode device configured to communicate wirelessly with at least one of the wireless electrode device and the external controller.

81. The method of claim 80, further comprising sequentially energizing the first wireless electrode device and the second wireless electrode device to create an oscillating electromagnetic field between the electrodes.

82. The method of claim 78, further comprising modifying at least one parameter of the nerve regeneration treatment based on the patient response.

83. The method of claim 78, wherein causing at least one of the first and second wireless electrode devices to administer a nerve regeneration treatment comprises causing the wireless electrode device to deliver a therapeutic electric signal to the damaged nerve.

84. The method of claim 78, wherein causing at least one of the first and second wireless electrode devices to administer a nerve regeneration treatment comprises causing the wireless electrode device to deliver a therapeutic fluid to the damaged nerve.

85. The method of claim 78, wherein at least one of the first and second wireless electrode devices includes at least one sensor.

86. The method of claim 78, further including detecting the patient response to the nerve regeneration treatment.

87. The method of claim 86, further comprising delivering a tagging agent proximate the damaged nerve.

88. The method of claim 87, further comprising measuring a growth of the damaged nerve by monitoring a position of the tagging agent over time.

89. A nerve regeneration system comprising:
a wireless electrode device implanted within a patient's body proximate a damaged nerve, the electrode device being configured to administer a nerve regeneration treatment to the damaged nerve and to detect a patient response to the nerve regeneration treatment; and
a controller located external to the patient's body and configured to wirelessly communicate with the electrode device.

90. The system of claim 89, further including a second wireless electrode device that differs in configuration with the wireless electrode device.

91. The system of claim 89, further comprising one or more second wireless electrode devices implanted within the patient's body and configured to wirelessly communicate with the controller.

92. The system of claim 91, wherein one or more of the second wireless electrode devices is configured to receive patient data from the wireless electrode device.

93. The system of claim 89, wherein the electrode device is configured to modify at least one parameter of the nerve regeneration treatment based on the detected patient response.

94. The system of claim 93, wherein the electrode device is configured to transmit the detected patient response to the controller, and the controller is configured to transmit a controlling signal to the electrode device to modify the at least one parameter based on the detected patient response.

95. The system of claim 89, wherein the electrode device is configured to provide an electric current to one or more additional electrode device.

96. The system of claim 89, wherein the controller comprises at least one of: a wireless communication device, a personal data assistant (PDA), and a wireless telephone.

97. The system of claim 89, wherein the electrode device comprises a fluid delivery system.

98. The system of claim 97, wherein the fluid delivery system is configured to deliver a therapeutic fluid to the damaged nerve.

99. The system of claim 89, wherein the electrode device comprises at least one sensor.

100. The system of claim 89, wherein the nerve generation treatment comprises an electric stimulation and the electrode device is configured to deliver an electric stimulation signal to the damaged nerve.

101. A method for treating a body comprising:
implanting an elongated tubular member in a patient's body proximate a damaged nerve, the elongated tubular member including a plurality of electrodes; and
energizing at least one of the electrodes to deliver an electric stimulation to a portion of the damaged nerve.

102. The method of claim 101, further comprising sequentially energizing the plurality of electrodes to create an oscillating electromagnetic field therebetween.

103. The method of claim 101, further comprising adjusting a parameter of the at least one of the electrodes to control the delivery of electric stimulation to the portion of the damaged nerve.

104. The method of claim 101, further comprising providing data indicative of the nerve's response to a controller coupled to the tubular member.

105. The method of claim 104, further comprising adjusting, by the controller, at least one parameter associated with the delivery of the electric stimulation to the portion of the damaged nerve based on the nerve's response.

106. The method of claim 105, wherein the at least one parameter includes one or more of: a field strength, a field direction, a current, and a voltage of the electric stimulation.

107. The method of claim 105, wherein the at least one parameter includes one or more of: a number, a sequence, or a combination of electrodes to be energized to deliver the electric stimulation.

108. The method of claim 101, further comprising injecting, by the controller, a therapeutic fluid into the tubular member.

109. The method of claim 101, wherein the tubular member comprises a bioabsorbable material.

110. The method of claim 101, wherein the tubular member includes a therapeutic fluid, the method further comprising delivering a therapeutic fluid to the damaged nerve.

111. The method of claim 110, wherein the therapeutic fluid comprises at least one of: a nerve growth agent, an anti-infection agent, and a pain reducing agent.

112. The method of claim 101, wherein implanting the tubular member further comprises securing a portion of the tubular member to the patient's body proximate a damaged nerve.

113. The method of claim 101, wherein the tubular member includes a hollow flexible mesh.

114. The method of claim 101, wherein the tubular member includes a polymeric foam material.

115. The method of claim 101, further comprising monitoring a nerve's response to the electric stimulation.

116. A nerve generation system, comprising:
an elongated tubular member configured to be implanted within a patient's body proximate a damaged nerve and configured to guide growth of the damaged nerve substantially therethrough; and
a plurality of electrodes disposed along a length of the tubular member,
wherein each of the electrodes is configured to deliver an electric stimulation to a portion of the damaged nerve.

117. The system of claim 116, further comprising a controller configured to communicate with at least one of the plurality of electrodes, wherein the controller is configured to control the delivery of the electric stimulation to the portion of the damaged nerve.

118. (canceled)

119. The system of claim 117, wherein the controller is in wireless communication with at least one of the plurality of electrodes.

120. The system of claim 117, wherein the controller is configured to provide electric energy to the plurality of electrodes.

121. The system of claim 117, wherein the controller is configured to monitor a signal indicative of the body's response to the delivered electric stimulation and adjust at least one parameter associated with the delivery of the electric stimulation based on the monitored signal.

122. The system of claim 121, wherein the at least one parameter comprises one or more of: a field strength, a field direction, a current, and a voltage of the electric stimulation.

123. The system of claim 121, wherein the at least one parameter comprises one or more of: a number, a sequence, or a combination of electrodes to be used for the electric stimulation.

124. The system of claim 117, wherein the controller comprises a fluid delivery device for injecting a therapeutic fluid into the tubular member.

125. The system of claim 124, wherein the controller is configured to adjust a delivery parameter associated with the delivery of the therapeutic fluid.

126. The system of claim 125, wherein the delivery parameter comprises one or more of a schedule, rate, or dosage of the therapeutic fluid.

127. The system of claim 116, wherein the plurality of electrodes comprises at least two electrodes each positioned at a proximal end and a distal end, respectively, of the tubular member.

128. The system of claim 116, wherein the tubular member comprises a bioabsorbable material.

129. The system of claim 116, wherein the tubular member is configured to deliver a therapeutic fluid to a portion of the damaged nerve.

130. The system of claim 129, wherein the therapeutic fluid is deposited in the tubular member configured to be released over time.

131. The system of claim 130, wherein the therapeutic fluid is coated at least partially on a surface of the tubular member.

132. The system of claim 129, wherein the therapeutic fluid comprises at least one of: a nerve growth agent; an anti-infection agent; and a pain reducing agent.

133. The system of claim 116, wherein the tubular member comprises a hollow flexible mesh structure.

134. The system of claim 116, wherein the tubular member comprises a polymeric foam material.

135. The system of claim 117, wherein the electrode is further configured to monitor the nerve's response to the electric stimulation.

136. A tissue manipulating device comprising:
a housing implanted in the body of a patient proximate a damaged nerve; and
an advanceable member at least partially disposed within the housing, the advanceable member being configured to advance from the housing to manipulate nerve tissue proximate the damaged nerve.

137. The device of claim 136, wherein the advanceable member comprises at least one projecting element.

138. The device of claim 137, wherein the at least one projecting element is operatively coupled to a drive member, the drive member being configured to extend the projecting element from the housing.

139. The device of claim 138, further comprising a controller disposed within the housing, the controller being configured to operate the drive member.

140. The device of claim 139, wherein the controller is configured to pulse the drive member to sequentially extend and retract the projecting element, thereby massaging nerve tissue proximate the damaged nerve.

141. The device of claim 139, wherein the controller is communicatively coupled to an external diagnostic tool.

142. The device of claim 139, therein the external diagnostic tool is configured to provide a command signal to the controller, wherein the command signal causes the controller to operate the drive member.

143. The device of claim 142, further including a sensor in data communication with the controller, the sensor being configured to monitor a nerve's response to the stimulation.

144. The device of claim 143, wherein the controller is further configured to provide data indicative of the nerve's response to the external diagnostic tool.

145. A tissue manipulating system comprising:
a sealed housing configured to be at least partially implanted within a body proximate a damaged nerve;
a fluid port in the sealed housing for receiving fluid;
an inflatable member in fluid communication with the fluid port; and
a controller configured to control flow of the fluid into and out of the inflatable member, thereby controlling inflation and deflation of the inflatable member.

146. The system of claim 145, wherein the inflatable member comprises a balloon.

147. The system of claim 145, wherein the controller comprises a syringe, and injecting the fluid through the syringe causes inflation of the inflatable member.

148. (canceled)

149. The system of claim 148, wherein the controller is configured to receive command signals from an external device.

150. The system of claim 145, further comprising a reservoir configured to store the fluid injected into the fluid port.

151. A method for treating a body comprising:
implanting a housing proximate a damaged nerve, the housing having at least one advanceable member at least partially disposed therein;
sequentially actuating the at least one advanceable member to stimulate the damaged nerve tissue; and
monitoring the damages nerve's response to the stimulation.

152. The method of claim 151, wherein the advanceable member comprises at least one projecting element coupled to a drive member, the method further comprising sequentially actuating the drive member to extend and retract the at least one projecting element into the damaged nerve.

153. The method of claim 151, wherein the advanceable member comprises an inflatable member coupled to a pump, the method further comprising sequentially operating the pump to control flow of the fluid into and out of the inflatable member, thereby controlling inflation and deflation of the inflatable member.

154. The method of claim 151, further comprising adjusting a parameter associated with the sequential actuation of the at least one advanceable member based on the damaged nerve's response to the stimulation.

155. The method of claim 154, wherein the parameter comprises at least one of a timing and a speed associated with the sequential actuation of the at least one advanceable member.

156. The method of claim 151, wherein the housing includes an electrode, the method further comprising delivering a therapeutic electric signal to the damaged nerve tissue.

157. The method of claim 151, wherein the housing includes a fluid delivery device, the method further comprising delivering a therapeutic fluid to the damaged nerve tissue.

158. A method for treating a body comprising:
depositing a magnetic therapeutic device proximate damaged nerve tissue, the magnetic therapeutic device comprising at least one electromagnet;
energizing the at least one electromagnet to create a stimulating magnetic field;
directing at least a portion of the magnetic field toward the damaged nerve tissue; and
monitoring the damaged nerve's response to the magnetic field.

159. The method of claim 158, wherein the at least one electromagnet includes a plurality of electromagnets, the method further comprising sequentially energizing the at least one electromagnet to create an oscillating magnetic field between the plurality of electromagnets.

160. The method of claim 158, wherein monitoring the damaged nerve's response to the magnetic field includes measuring a growth of the damaged nerve.
